(19) United States
(12) Patent Application Publication
(10) Pub. No.: US 2022/0371973 A1
(43) Pub. Date: Nov. 24, 2022

(54) Method and Molecules

(71) Applicant: MedImmune, LLC, Gaithersburg, MD (US)

(72) Inventors: Jia Lin, Gaithersburg, MD (US); Ronald James Christie, Gaithersburg, MD (US); Changshou Gao, Gaithersburg, MD (US)

(21) Appl. No.: 17/622,387

(22) PCT Filed: Jul. 2, 2020
(86) PCT No.: PCT/US2020/040675

§ 371 Date: Dec. 23, 2021

Related U.S. Application Data
(60) Provisional application No. 62/870,895, filed on Jul. 5, 2019.

(51) Int. Cl. C07C 13/08; C07K 16/28; C07K 14/00

(52) U.S. Cl. CPC C07C 13/08; C07K 16/2803; C07K 14/00; C07K 2317/51

(57) ABSTRACT

The present invention provides conjugates of a first polypeptide and a second polypeptide wherein the link between the first polypeptide and the second polypeptide comprises the following moiety.

The present disclosure relates to a method of conjugating two polypeptides and a molecule made by said method.

### BACKGROUND

The use of Diels-Alder reactions between dienes and dienophiles to conjugate a biological molecule to a payload is described in WO 2018/218093. The approach is presented as being able to be carried out under mild conditions, sometimes in the absence of additional reagents.

It has been surprisingly found by using certain moieties which are disclosed in WO 2018/218093, which can act as both a diene and a dienophile, proximity-driven reactions can occur that can be used to link two polypeptides together.

Several strategies to enable unnatural proximity-dependent reactivity in proteins have also been described, capitalizing on a complementary pair of canonical and noncanonical amino acids to covalently link protein units together. These pairs include noncanonical amino acids that bear thiol-reactive bromo, fluoro, or fluorobenzene functional groups (Cigler 2017, Xiang 2013, Embaby 2018); lysine-reactive aryl carbamate groups (Xuan 2017); and lysine-, histidine-, and tyrosine-reactive fluorosulfate groups (Wang 2018). The general coupling strategy employed in these systems is to introduce a single noncanonical amino acid into the protein sequence along with the appropriate canonical amino acid reaction partner in close proximity in the folded or assembled protein structure.

### SUMMARY OF THE DISCLOSURE

A first aspect of the present invention provides a conjugate of a first polypeptide and a second polypeptide wherein the link between the first polypeptide and the second polypeptide comprises the moiety:

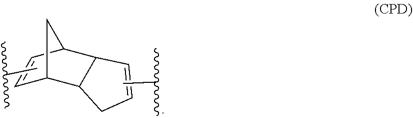

A second aspect of the present invention provides a method of conjugating a first polypeptide and a second polypeptide wherein the first polypeptide and the second polypeptide each comprises the moiety:

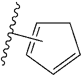

(cyclopentadienyl, CP), where the conjugating involves a Diel-Alder reaction between the cyclopentadienyl moieties.

The Diels-Alder reaction as employed herein refers to a 4 plus 2 cycloaddition reaction which forms a cyclohexene ring, which is a part of a fused ring system.

Surprisingly the present inventors have established that the two cyclopentadienyl groups undergo such a Diels-Alder reaction when in proximity under mild conditions. In other instances, the bioconjugation reaction can occur in one-step, without need for additional reagents other than the two polypeptides and solvent.

Furthermore, reactive crosslinkers and non-natural amino acids comprising cyclopentadienyl groups are synthetically accessible and can be produced in high yields in simple and straightforward routes, as described in WO 2018/218093.

Incorporation of Cyclopentadienyl Group into Polypeptides

The cyclopentadienyl group may be incorporated into the first and second polypeptides via the addition of a linker or by incorporating a non-natural amino acid into the polypeptide sequence, as described in WO 2018/218093.

In one embodiment at least one of the cyclopentadienyl groups is contained in a non-natural amino acid, for example a non-natural amino acid derived from lysine, cysteine, selenocysteine, aspartic acid, glutamic acid, serine, threonine, glycine, and tyrosine.

In one embodiment at least one of the cyclopentadienyl group is in a side chain of the amino acid.

In one embodiment the non-natural amino has a formula (I):

R^{X}—X¹—O₀₋₁C(O)-amino-acid-residue (I)
wherein:
R^{x} represents

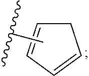

and
X¹ represents
   i) a saturated or unsaturated branched or unbranched C₁₋₃ alkylene chain, wherein at least one carbon (for example 1, 2 or 3 carbons) is replaced by a heteroatom selected from O, N, S(O)₀₋₃, wherein said chain is optionally, substituted by one or more groups independently selected from oxo, halogen, amino, —C₁₋₃alkylene-N₃, or —C₂₋₅alkynyl; or
   ii) together with a carbon from the carbocyclyl or heterocyclyl represents a cyclopropane ring linked to a saturated or unsaturated (in particular saturated) branched or unbranched C₁₋₆ alkylene chain, wherein at least one carbon (for example 1, 2 or 3 carbons) is replaced by a heteroatom selected from O, N, S(O)₀₋₃, wherein said chain is optionally, substituted by one or more groups independently selected from oxo, halogen, amino, —C₁₋₃alkylene-N₃, or —C₂₋₅alkynyl; and —O₀₋₁C(O)— is linked through a side chain of an amino acid.

In one embodiment the non-natural amino acid is a residue of the structure of formula (II):

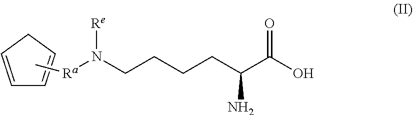

wherein R^{a} represents:

i) a saturated or unsaturated branched or unbranched C₁₋₈ alkylene chain, wherein at least one carbon (for example 1, 2 or 3 carbons) is replaced by a heteroatom selected from O, N, S(O)₀₋₃, wherein said chain is optionally, substituted by one or more groups independently selected from oxo, halogen, amino; or

ii) together with a carbon from the 5 membered ring represents a cyclopropane ring linked to a saturated or unsaturated (in particular saturated) branched or unbranched C₁₋₆ alkylene chain, wherein at least one carbon (for example 1, 2 or 3 carbons) is replaced by a heteroatom selected from O, N, S(O)₀₋₃, wherein said chain is optionally, substituted by one or more groups independently selected from oxo, halogen, amino;

R^{e} represents H, saturated or unsaturated (in particular saturated) branched or unbranched C₁₋₈ alkylene chain, wherein one or more carbons are optionally replaced by —O— and the chain is optionally substituted by one or more halogen atoms (such as iodo), N₃ or —C₂₋₅ alkynyl.

In one embodiment R^{a} is —(CH₂)ₘC(O)—, —CH₂(CH₃)C(O)—, —(CH₂)ₘCH₂OC(O)—, —CHCHCH₂OC(O)—, or —OCH₂CH₂CC(O)— and m represents 0 or 1.

In one embodiment R^{e} represents H or —CH₂OCH₂CH₂N₃.

In one embodiment the non-natural amino acid is a residue of the structure of formula (IIa):

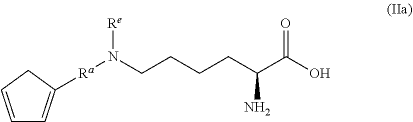

wherein R^{a}, R^{e} and X² are defined above.

In one embodiment the non-natural amino acid has the structure of formula (IIb):

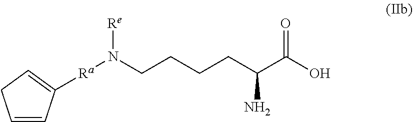

wherein R^{a}, R^{e} and X² are defined above.

Generally compounds, for example formula (I), (II), (IIa) and (IIb) will at most contain only one azide group.

In one embodiment the non-natural amino acid is selected from the group comprising:

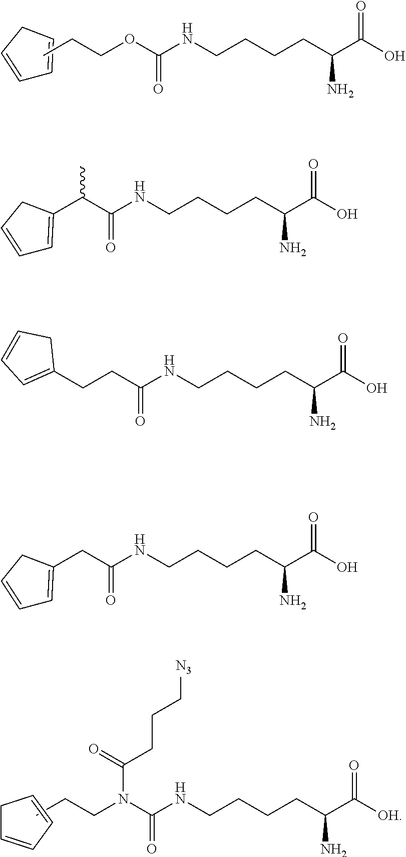

Thus, in one embodiment at least one link between the group CPD and the polypeptide may be of the formula (I*):

^{CPD}*—X¹—O₀₋₁C(O)—*^{PP} (I*)
wherein:
^{CPD}* represents where the link is joined to CPD;
*^{PP} represents where the link is join to the polypeptide; and
X¹ and —O₀₋₁C(O)— are as defined for formula I.

Thus, in one embodiment at least one link between the group CPD and the polypeptide may be of the formula (II*):

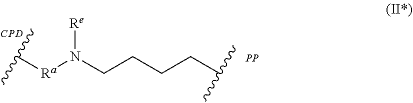

^{CPD}* represents where the link is joined to CPD;
*^{PP} represents where the link is join to the polypeptide; and
R^{a} and R^{e} are as defined for formula (II).

In one embodiment, CPD and the link may be of formula (IIa-CPD):

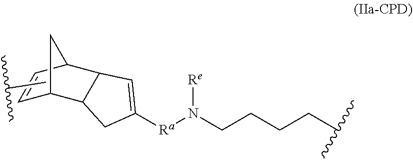

wherein R^{a} and R^{e} are defined above.

In one embodiment, CPD and the link may be of formula (IIb-CPD):

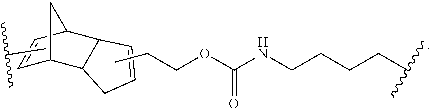

wherein R^{a} and R^{e} are defined above.

Generally the groups of formula (I*), (II*), (IIa-CPD) and (IIb-CPD) will at most contain only one azide group.

In one embodiment CPD and the link to the polypeptide is selected from the group comprising:

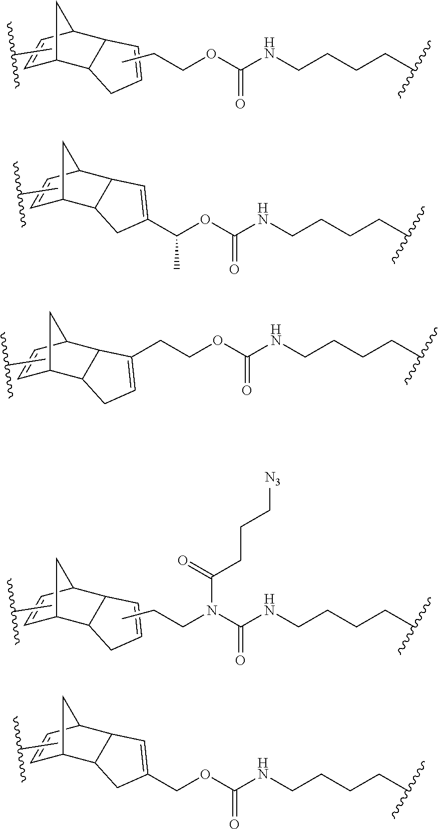

In one embodiment the cyclopentadienyl group is incorporated into the first and/or second polypeptides via the addition of a linker to an amino acid residue in the first and/or second polypeptide, for example where the amino acid is a cysteine or lysine.

In one embodiment the cyclopentadienyl group containing molecule before addition to said amino acid residue in the polypeptide has the structure of formula (III):

R^{X}—Bₙ—X³ₘ—Yₚ—Z (III)
wherein
n represents 0 or 1;
m represents 0 or 1;
p represents 0 or 1;
R^{x} represents

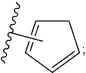

and
B represents C₁₋₆ alkylene, —C₃₋₄ cycloalkylC₁₋₆ alkylene-; wherein a optionally a sugar residue (such as glucose, glucosamine, galactose, galactosamine, lactose, mannose, and fructose) is contained in the alkylene chain of any one of the same, and wherein the alkylene chain of any one of said variables defined for B bears optionally bears one or two substituents independently selected from an N- and O-linked sugar residue (such as glucose, glucosamine, galactose, galactosamine, lactose, mannose, and fructose):
X³ represents —(R¹)NC(O)—, —C(O) N(R¹)—, —OC(O)—, —OC(O)N—;
R¹ represents H or —CH₂OCH₂CH₂R²;
R² represents —N₃, C₂₋₅ alkynyl, or halogen, such as iodo;
Y represents —(OCH₂)_{q}C₂₋₆alkylene, or —C₂₋₆ alkylene optionally substituted with —NR³R⁴,
wherein q is 1 to 7000;
R³ and R⁴ independently represents H or C₁₋₃ alkyl;
Z represents —C(O)OR⁵, R^{5′}, —NC(O)R⁶, —C₂₋₅alkylene, CH₂—O—NH₂ or halogen such as iodo;
R⁵ represents C₁₋₆ alkyl, succinimide, C₆F₄H (tetrafluorohexyl), or H;
R^{5′} represents a sulfur bridging group, for example a dibromomaleimide, a dichloroacetone, a divinyl pyridine or a derivative of any one of the same,
R⁶ represents:

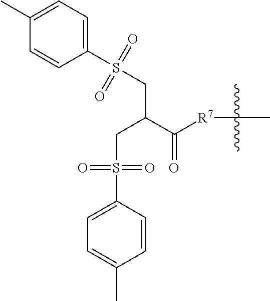

wherein
R⁷ is C₁₋₆ alkylene optionally bearing one or more (such as one, two or three) groups selected from hydroxyl, sulfo, amino and —(OCH₂)ᵥC₂₋₆alkylene, and phenyl optionally bearing one or more (such as one, two or three) groups selected from hydroxyl, sulfo, amino and —(OCH₂)ᵥC₂₋₆alkylene;
v is an integer 1, 2, 3, 4 or 5
[symbol] represents where the fragment is connected to the rest of the molecule.

In one embodiment the cyclopentadienyl group containing molecule has a structure:

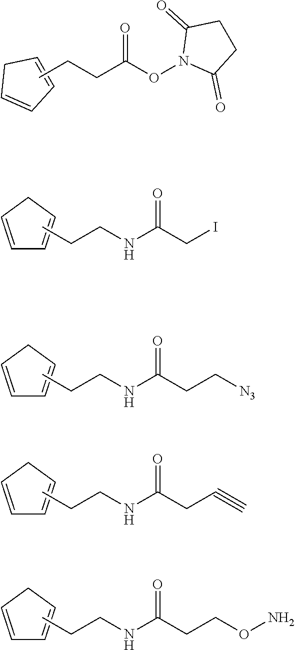

Thus, in one embodiment at least one link between the group CPD and the polypeptide may be of the formula (III*):

^{CPD}*—Bₙ—X³ₘ—Yₚ—Z*—*^{PP} (III*)
wherein:
^{CPD}* represents where the link is joined to CPD;
*^{PP} represents where the link is join to the polypeptide; and
B, X³, Y, n, m and p are as defined for formula III, and Z* is the residue of Z on reaction with the polypeptide. Therefore, in some embodiments Z* can be, for example, —C(O)O—, —NC(O)—, triazolyl, —S—, or —NHC(O)—.

In one embodiment the group III* has a structure:

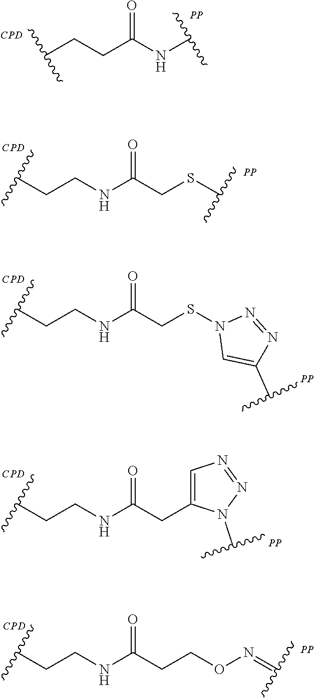

In some embodiments, the link between the group CPD and the first polypeptide and the link between the group CPD and the second polypeptide may be of the same nature, for example, may be of the same generic formula. In some of these embodiments, the two links may be the same.

In one embodiment of the method, the reaction is performed at a temperature in the range 0° C. to 70° C. The minimum temperature for the method may be 0° C., 5° C., 10° C., 15° C. or 20° C. The maximum temperature for the method may be 70° C., 60° C., 50° C., 40° C., 30° C. or 25° C. In some embodiments, the method may be carried out at ambient temperature.

In one embodiment of the method, the polypeptide is subjected to freeze-thaw cycles

In one embodiment the reaction is performed in aqueous solvent, for example aqueous organic solvent systems, a buffer such as PBS optionally comprising a polar aprotic solvent, such as DMSO or a surfactant, such as polysorbate 80 or combinations thereof.

### BRIEF SUMMARY OF THE FIGURES

FIG. 1. Production of antibodies incorporating cyclopentadiene in the human IgG1 antibody Fc region. FIG. 1A. The structure of CP1 nnAA (also termed CpK) and the 1C1 antibody used to demonstrate CP1 nnAA incorporation. FIG. 1B. Process for antibody expression. FIG. 1C. Distance between amino acid α-carbons and orientation of serine and lysine side chains at positions S239 and K274 in an assembled antibody structure. Amino acid α-carbons are shown as colored spheres and approximate orientation of native lysine and serine side chains are shown as yellow arrows. The antibody Fab region is not shown. Only the 2-substituted cyclopentadiene is shown.

FIG. 2. Characterization of reduced monoclonal antibody (mAb) products bearing CpK by SDS-PAGE. FIG. 2A. Reduced SDS-PAGE analysis of 1C1 antibodies bearing CpK at the positions specified in the legend. FIG. 2B. More detailed view of reduced SDS-PAGE analysis of 1C1 antibodies bearing CpK at position S239 (1) or K274 (2). WT—wild-type 1C1 mAb.

FIG. 3. Deglycosylated mass spectrometry analysis of antibody products. FIG. 3A. 1C1 wild-type mAb, intact. FIG. 3B. 1C1.S239CP1 mAb, intact. FIG. 3C. 1C1.K274CP1 mAb, intact. FIG. 3D. 1C1 wild-type mAb, reduced. FIG. 3E. 1C1.S239CP1 mAb, reduced. FIG. 3F. 1C1.K274CP1 mAb, reduced. FIG. 3G. Deglycosylated mass spectrometry analysis of 1C1.P232CP1 mAb.

FIG. 4. Evaluation of the CpK Diels-Alder adduct in 1C1 antibody bearing CpK at position S239 (1). FIG. 4A. Enzymatic digestion of 1 to generate a peptide fragment containing S239CpK. FIG. 4B. Reverse phase high performance liquid chromatography analysis (RP-HPLC) analysis of the digestion product showing the extracted ion chromatogram at the m/z of the peptide fragment. FIG. 4C-D. Mass spectrometry (MS) analysis of monomer and dimer peptide fragments with the ionization states indicated. FIG. 4E-F. Zoomed mass spectra of the 497 amu peak common to both monomer and dimer peptide fragments. FIG. 4G. Tandem mass spectrometry (MS/MS) analysis of the peptide dimer fragment. Note that the carbamate of CpK was cleaved back to lysine under these conditions. Peptide containing degraded lysine and the proposed Diels-Alder adduct are indicated with *.

FIG. 5A. MS/MS spectra of monomer peptide fragment of 1 generated by digestion with IdeS and trypsin. Note that the carbamate of CpK was cleaved back to lysine under these conditions. Intact peptide containing degraded lysine and the proposed Diels-Alder adduct are indicated with *.

FIG. 5B. MS/MS spectra of dimer peptide fragment of 1 generated by digestion with IdeS and trypsin. Note that the carbamate of CpK was cleaved back to lysine under these conditions. Intact peptide containing degraded lysine and the proposed Diels-Alder adduct are indicated with *.

FIG. 6. Overview of the MAB1VV.K409R antibody constructs used to screen amino acid positions for their ability to form Diels-Alder crosslinks. MAB1VV.K409R is a monovalent monospecific IgG1 antibody.

FIG. 7. Reduced SDS-PAGE analysis of MAB1VV.K409R antibodies bearing CP1 nnAA at the specified positions.

FIG. 8. Overview of engineered features to generate MAB2+MAB1 monovalent bispecific antibody comprising a heavy chain heterodimer covalently linked by a Diels-Alder adduct formed between CP1 nnAAs.

FIG. 9. Overview of methods to generate antibodies comprising heterogenous heavy-chains covalently linked by a Diels-Alder adduct. FIG. 9A. Cotransfection method. FIG. 9B. Coculture method.

FIG. 10. Reduced SDS-PAGE analysis of bispecific antibody product after sequential purification with KappaSelect beads and LambdaSelect beads. The heterodimer heavy chain antibody was generated by the cotransfection method.

FIG. 11. Reduced deglycosylated mass spectrometry analysis of antibody products following purification by protein A beads. FIG. 11A. MAB2RR.S239CP1.F405R antibody. FIG. 11B. MAB1 EE.S239CP1. F405L antibody. FIG. 11C. MAB2RR.S239CP1.F405R+MAB1 EE.S239CP1. F405L bispecific antibody produced by the cotransfect method. FIG. 11D. MAB2RR.S239CP1. F405R+MAB1 EE.S239CP1. F405L bispecific antibody produced by the coculture method. Spectra show the light chain (LC), heavy chain (HC), and heavy-chain dimer regions (˜20-100 KDa).

FIG. 12. Reduced deglycosylated mass spectrometry analysis of antibody heavy chain dimer products. FIG. 12A. MAB2RR.S239CP1.F405R antibody. FIG. 12B. MAB1EE.S239CP1.F405L antibody. FIG. 12C. MAB2RR.S239CP1.F405R+MAB1EE.S239CP1.F405L bispecific antibody produced by the cotransfect method. FIG. 12D. MAB2RR.S239CP1. F405R+MAB1 EE.S239CP1. F405L bispecific antibody produced by the coculture method. Spectra are zoomed in to show the heavy-chain dimer region (˜100 KDa).

FIG. 13. Reduced RP-HPLC analysis of antibody products, demonstrating that CP1 nnAA heavy chain dimer formed. Solid lines represent homodimer antibodies and the dashed line represents bispecific heterodimer antibody produced by the cotransfection method.

FIG. 14. Analysis of antigen binding by Octet measurement with signals normalized prior to first antigen binding, demonstrating that first antigen binding is maintained in bispecific antibodies produced with CP1 nnAA. A) MAB1EE.S239CP1.F405L antibody, B) MAB2RR. S239CP1.K409R antibody, C) MAB1EE.S239CP1.F405L+MAB2RR.S239CP1.K409R bispecific antibody produced by cotransfection and purified by LambdaSelect beads, D) MAB1EE.S239CP1.F405L+MAB2RR.S239CP1.K409R bispecific antibody produced by coculture and purified by LambdaSelect beads, E) MAB1EE.S239CP1.F405L+MAB2RR.S239CP1.K409R bispecific antibody produced by cotransfection and purified by LambdaSelect beads followed by KappaSelect beads, F) MAB1EE.S239CP1.F405L+MAB2RR.S239CP1.K409R bispecific antibody produced by coculture and purified by LambdaSelect beads followed by KappaSelect beads, G) Positive control antibody, H) non-binding isotype control antibody.

FIG. 15. Analysis of antigen binding by Octet measurement with signals normalized prior to second antigen binding, demonstrating that second antigen binding is maintained in bispecific antibodies produced with CP1 nnAA. A) MAB1EE.S239CP1.F405L antibody, B) MAB2RR. S239CP1.K409R antibody, C) MAB1EE.S239CP1.F405L+MAB2RR.S239CP1.K409R bispecific antibody produced by cotransfection and purified by LambdaSelect beads, D) MAB1EE.S239CP1.F405L+MAB2RR.S239CP1.K409R bispecific antibody produced by coculture and purified by LambdaSelect beads, E) MAB1EE.S239CP1. F405L+MAB2RR.S239CP1. K409R bispecific antibody produced by cotransfection and purified by LambdaSelect beads followed by KappaSelect beads, F) MAB1 EE.S239CP1.F405L+MAB2RR.S239CP1.K409R bispecific antibody produced by coculture and purified by LambdaSelect beads followed by KappaSelect beads, G) positive control bispecific antibody, H) non-binding isotype control antibody.

### DETAILED DISCLOSURE

Conjugation (reaction) as employed herein is a simply a reaction linking a molecule to another entity. In the context of the present specification a first polypeptide conjugated to a second polypeptide is the product obtained from a conjugation reaction.

An amino acid residue as employed herein refers to a natural or non-natural amino acid linked, for example to another amino acid, via the N and/or C terminal of the amino acid, in particular where at least one link is a peptide bond.

A non-natural amino acid as employed herein refers to an amino acid which is other than one of the twenty-one naturally occurring amino acids. For example the non-natural amino acid may comprises cyclopentadienyl group and also contains the amino and carboxylic functional groups in the relative positions that are characteristic of natural amino acids. Certain non-natural amino acids and methods for making the same are disclosed in WO2015/019192, incorporated herein by reference.

The non-natural amino acids are generally derived from natural amino acid. Derived from a natural amino acid refers to the fact that the non-natural amino acid is based on (or incorporates) or is similar to the structure of natural amino acid, for example the alkylene chain in lysine may be shortened to provide a 3 carbon chain as opposed to the natural 4 carbon chain but the structural relationship or similarity to lysine still exists. Thus derivatives of natural amino acids include modifications such as incorporating the diene or dienophile, lengthening or shortening an alkylene chain, adding one or more substituents to a nitrogen, oxygen, sulfur in a side chain or converting a nitrogen, oxygen or sulfur into a different functional group or a combination of any of the same. Usually the majority of modifications will be the addition of structure in the non-natural amino acid. However, modification may include removed or replacing an atom naturally found in an amino acid.

Natural amino acid as employed herein refers to the 21 proteinogenic amino acids (namely arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan).

In one embodiment the non-natural amino acid comprising the cyclopentadienyl group is incorporated in the amino acid sequence of the first and/or second polypeptide, for example in the expression process of a recombinant polypeptide. This is advantageous because it locates the amino acid is precisely position, which then facilitates a very specific conjugation between the first and second polypeptide.

In one embodiment the non-natural amino acid may be appended to the first and/or second polypeptide via a linker and conjugation reaction.

In one embodiment the non-natural amino acid of the present disclosure is derived from lysine asparagine, glutamine, cysteine, selenocysteine, aspartic acid, glutamic acid, serine, threonine, glycine and tyrosine.

In one embodiment the polypeptide is engineered to remove one or more lysine residues from the original or native sequence.

Where the cyclopentadienyl group is introduced into the polypeptide via a linker, functionality such as N₃, halo, succinimide or an alkyne can be reacted with, for example lysine in the amino acid sequence of the polypeptide.

Alkyl as used herein refers to straight chain or branched chain alkyl, such as, without limitation, methyl, ethyl, n-propyl, iso-propyl, butyl, n-butyl and tert-butyl. In one embodiment alkyl refers to straight chain alkyl.

Amino as employed herein refers to —NH₂, C₁₋₄ mono or di-acyl amino is intended to refer to —NHC(O)C₁₋₃ alkyl and to (—NC(O)C₃ alkyl) C(O)C₁₋₃ alkyl) respectively.

C₁₋₄ mono or di-alkyl amino is intended to refer to —NHC₁₋₄ alkyl and —N(C₁₋₄ alkyl) (C₁₋₄ alkyl) respectively.

Halogen or halo includes fluoro, chloro, bromo or iodo, in particular fluoro, chloro or bromo, especially fluoro or chloro.

Oxo as used herein refers to C═O and will usually be represented as C(O).

Alkylene as employed herein refers to branched or unbranched carbon radicals, such as methylene (—CH₂—) or chains thereof.

C₂₋₅ alkyne as employed herein refer to a group or radical containing a triple bond and between two and 5 carbon atoms in a linear or branched arrangement. In one embodiment only substituent in the molecule or fragment comprises an alkyne.

In relation to a saturated or unsaturated, branched or unbranched C₁₋₈ alkyl chain, wherein at least one carbon (for example 1, 2 or 3 carbons, suitably 1 or 2, in particular 1) is replaced by a heteroatom selected from O, N, S(O)₀₋₃, wherein said chain is optionally, substituted by one or more groups independently selected from oxo, halogen, it will be clear to persons skilled in the art that the heteroatom may replace a primary, secondary or tertiary carbon, that is CH₃, —CH₂— or a —CH— or a branched carbon group, as technically appropriate. N₃ as employed herein refers to an azide.

Sulfo as employed herein refers to a sulphur atom bonded to one, two or three oxygen 25 atoms.

Suitable sugars for addition to compounds of formula (III) include glucose, glucosamine, galactose, galactosamine, mannose, fructose, galactose, maltose and lactose. Advantageously the addition of a sugar molecule may increase solubility.

Polypeptides for Use in the Present Disclosure

The terms “polypeptide”, “peptide”, and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component.

Also included within the definition are, for example, polypeptides containing one or more analogs of an amino acid (including, for example, unnatural amino acids, etc.), as well as other modifications known in the art. This is understood because the polypeptides of the instant disclosure may be based upon antibodies, such as being fragments thereof.

Polypeptide as employed herein refers to a sequence of 5 or more amino acids, with or without secondary or tertiary structure. Thus in the present disclosure the term “polypeptides” includes peptides, polypeptides and proteins. These are used interchangeably unless otherwise specified.

In one embodiment the polypeptide is a protein. Proteins generally contain secondary and/or tertiary structure and may be monomeric or multimeric in form. In one embodiment the first and second polypeptides are the same, i.e. both comprise amino acid sequences that are identical, such that the first and second polypeptides form a homodimer. In one embodiment the first and second polypeptides are different, i.e. both comprise amino acid sequences that are different to one another, such that the first and second polypeptides form a heterodimer.

The first and second polypeptides contemplated for use in the present disclosure are not particularly limited and can be any pair of polypeptides that are intended to be covalently linked together. In one embodiment the first and second polypeptide are known or believed to associate non-covalently or covalently and this association brings the CP moieties present on the first and second polypeptides together such that the CP moieties undergo a Diels-Alder (DA) reaction and form a covalent linkage that comprises the CPD moiety.

The CP moieties may be located at a first amino acid residue in the first polypeptide and at a second amino acid residue in the second polypeptide, where the first and second amino acid residues are at a sufficient proximity in the assembled protein structure. The assembled protein structure comprises the first polypeptide in covalent or non-covalent association with the second polypeptide, and typically does not contain the link comprising the CPD moiety. Distances between the first and second amino acid α-carbons and the orientation of the side chains in the assembled protein structure amino acid α-carbons can be calculated from a three-dimension structure of the first polypeptide associated with the second polypeptide, for example an X-ray crystal structure or a NMR structure. For example, distances and orientation can be estimated using the PyMOL software as described in the examples. Where the first and second polypeptide form an antibody molecule or part of an antibody molecule, the assembled protein structure may be the crystal structure from PDB entry 1FC1 (version 1.2).

Alternatively or additionally, the CP moieties may be located at a first amino acid residue in the first polypeptide and at a second amino acid residue in the second polypeptide, where the side chains of the native amino acids at these positions are orientated towards each other in the assembled protein structure. The side chain of the native amino acid in this context means the side chain that is present at the amino acid position before the CP moiety is incorporated. For example, where the CP moiety is incorporated by the addition of a linker to a lysine or a cysteine, the side chain of the native amino acid may be the lysine or cysteine. As another example, where the CP moiety is contained in a non-natural amino acid, the side chain of the native amino acid may be the natural amino acid that was present at the position where the non-natural amino acid is incorporated. As described above, the orientation of side chains can be determined using the PyMOL software as described in the examples.

Amino acids may be considered at a sufficient proximity if the distance between the amino acid α-carbons of the first and second amino acids is less than 50 Å, less than 45 Å, less than 40 Å, less than 35 Å, less than 30 Å, less than 25 Å, less than 24 Å, less than 23 Å, less than 22 Å, less than 21 Å, or less than 20 Å in the assembled protein structure, optionally where the assembled protein structure is a crystal structure.

Amino acids may be considered to be at a sufficient proximity if the distance between the amino acid α-carbons of the first and second amino acids is greater than 5 Å, greater than 6 Å, greater than 7 Å, greater than 8 Å, greater than 9 Å, greater than 10 Å, greater than 11 Å, greater than 12 Å, greater than 13 Å, greater than 14 Å, or greater than 15 Å in the assembled protein structure, optionally where the assembled protein structure is a crystal structure.

Amino acids may be considered to be at a sufficient proximity if the distance between the α-carbons of the first and second amino acids is between 5 Å and 50 Å, between 5 Å and 45 Å, between 5 Å and 40 Å, between 5 Å and 35 Å, between 5 Å and 30 Å, between 5 Å and 25 Å, between 5 Å and 20 Å, between 6 Å and 50 Å, between 6 Å and 45 Å, between 6 Å and 40 Å, between 6 Å and 35 Å, between 6 Å and 30 Å, between 6 Å and 25 Å, between 6 Å and 20 Å, between 7 Å and 50 Å, between 7 Å and 45 Å, between 7 Å and 40 Å, between 7 Å and 35 Å, between 7 Å and 30 Å, between 7 Å and 25 Å, between 7 Å and 20 Å, between 8 Å and 50 Å, between 8 Å and 45 Å, between 8 Å and 40 Å, between 8 Å and 35 Å, between 8 Å and 30 Å, between 8 Å and 25 Å, between 8 Å and 20 Å, between 9 Å and 50 Å, between 9 Å and 45 Å, between 9 Å and 40 Å, between 9 Å and 35 Å, between 9 Å and 30 Å, between 9 Å and 25 Å, between 9 Å and 20 Å, between 10 Å and 50 Å, between 10 Å and 45 Å, between 10 Å and 40 Å, between 10 Å and 35 Å, between 10 Å and 30 Å, between 10 Å and 25 Å, between 10 Å and 20 Å, between 11 Å and 50 Å, between 11 Å and 45 Å, between 11 Å and 40 Å, between 11 Å and 35 Å, between 11 Å and 30 Å, between 11 Å and 25 Å, between 11 Å and 20 Å, between 12 Å and 50 Å, between 12 Å and 45 Å, between 12 Å and 40 Å, between 12 Å and 35 Å, between 12 Å and 30 Å, between 12 Å and 25 Å, between 12 Å and 20 Å, between 13 Å and 50 Å, between 13 Å and 45 Å, between 13 Å and 40 Å, between 13 Å and 35 Å, between 13 Å and 30 Å, between 13 Å and 25 Å, between 13 Å and 20 Å, between 14 Å and 50 Å, between 14 Å and 45 Å, between 14 Å and 40 Å, between 14 Å and 35 Å, between 14 Å and 30 Å, between 14 Å and 25 Å, between 14 Å and 20 Å, between 15 Å and 50 Å, between 15 Å and 45 Å, between 15 Å and 40 Å, between 15 Å and 35 Å, between 15 Å and 30 Å, between 15 Å and 25 Å, or between 15 Å and 20 Å in the assembled protein structure, optionally where the assembled protein structure is a crystal structure.

In some embodiments where the first and second polypeptides are different, the CP moiety is located at a position in the first and/or second polypeptide that is not expected to undergo a Diels-Alder reaction to form a homodimeric conjugate between two copies of the first polypeptide, or two copies of the second polypeptide. In other words, the positions of the CP moiety may discourage the formation of a homodimeric conjugate. For example, the CP moiety may be located at an amino acid residue in the first polypeptide, such that in an assembled protein structure comprising two copies of the first polypeptide, the side chains of the native amino acids at these positions are orientated away from each other and/or the α-carbons are not in sufficient proximity. Similarly, the CP moiety may be located at an amino acid residue in the second polypeptide, such that in an assembled protein structure comprising two copies of the second polypeptide, the side chains of the native amino acids at these positions are orientated away from each other and/or the α-carbons are not in sufficient proximity.

Particular examples of the polypeptides that are contemplated for use in the present disclosure are described below.

In one embodiment the first or second polypeptide is or comprises a binding member. In another embodiment the first and second polypeptide form a binding member or part of a binding member when conjugated. In this context, a “binding member” is a polypeptide or protein that specifically binds a target molecule. The term “specific” may refer to the situation in which the binding member will not show any significant binding to molecules other than its specific target molecule(s). Such molecules are referred to as “non-target molecules”.

In some embodiments, the binding member is considered to not show any significant binding to a non-target molecule if the extent of binding to a non-target molecule is less than about 10% of the binding of the binding molecule to the target as measured, e.g., by ELISA, SPR, Bio-Layer Interferometry (BLI), MicroScale Thermophoresis (MST), or by a radioimmunoassay (RIA). Alternatively, the binding specificity may be reflected in terms of binding affinity, where the binding member described herein binds to its target molecule with an affinity that is at least 0.1 order of magnitude greater than the affinity towards another, non-target molecule. In some embodiments, the binding member binds to its target molecule with an affinity that is one of at least 0.2, 0.3, 0.4, 0.5, 0.6, 0.7, 0.8, 0.9, 1.0, 1.5, or 2.0 orders of magnitude greater than the affinity towards another, non-target molecule.

Binding members include for example antibody molecules, engineered protein domains such as scaffold, aptamers and designed ankyrin repeat proteins (DARPins).

In one embodiment the binding member is an antibody molecule.

Antibody molecule as employed herein is a generic term referring to antibodies, antibody binding fragments and antibody formats such as bispecific or multispecific antibodies comprising said antibodies or binding fragments thereof. The antibody molecule may be human or humanised. The antibody molecule may be a polyclonal or a monoclonal antibody molecule.

As used herein, the term “antibody molecule” encompasses intact polyclonal antibodies, intact monoclonal antibodies, antibody fragments (such as Fab, Fab′, F(ab′)2, and Fv fragments), single chain antibody fragments (scFv and disulfide stabilized scFv (dsFv)), multispecific antibodies such as bispecific antibodies generated from at least two different antibodies or multispecific antibodies formed from antibody fragments (see, e.g., PCT Publications WO96/27011, WO2007/024715, WO2009018386, WO2009/080251, WO2013006544, WO2013/070565, and WO2013/096291), chimeric antibodies, humanized antibodies, human antibodies, fusion proteins comprising an antigen-binding fragment of an antibody, such as an scFv-Fc fusion, and any other modified immunoglobulin molecule comprising an antigen-binding fragment so long as the antibody molecules exhibit the desired biological activity.

Antibody molecules and methods for their construction and use are well-known in the art and are described in, for example, Holliger 2005. It is possible to take monoclonal and other antibody molecules and use techniques of recombinant DNA technology to produce other antibody or chimeric molecules which retain the specificity of the original antibody. Such techniques may involve introducing CDRs or variable regions of one antibody molecule into a different antibody molecule (EP-A-184187, GB 2188638 Å and EP-A-239400).

A typical antibody comprises at least two heavy (H) chains and two light (L) chains interconnected by disulfide bonds. Disulfide bonds that connect the two heavy chains to each other and connect heavy chains to the light chains are termed “inter-chain” disulfide bonds. Each heavy chain is comprised of a heavy chain variable region (abbreviated herein as VH, VH region, or VH domain) and a heavy chain constant region. The heavy chain constant region is comprised of three or four constant domains, CH1, CH2, CH3, and CH4. A hinge region is located between the CH1 and CH2 domain. The Fc region includes the polypeptides comprising the constant region of an antibody excluding the first constant region immunoglobulin domain, and fragments thereof. Thus, for IgG the “Fc region” refers to CH2 and CH3 and optionally all or a portion of the flexible hinge region N-terminal to these domains. The term “Fc region” can refer to this region in isolation, or this region in the context of an antibody, antibody fragment, or Fc fusion protein.

The location of the constant domains can be defined according to EU numbering (Edelman 1969). The CH1 domain may be located between positions 114-215, the hinge region located between positions 216-230, the CH2 domain located between positions 231-340 and the CH3 domain located between positions 341-447, wherein the amino acid residue positions are numbered according to EU numbering.

In some embodiments the CH region is human immunoglobulin G1 constant (IGHG1; UniProt: P01857-1, v1) or a fragment thereof. Positions 1 to 98 of P01857 form the CH1 domain. Positions 99 to 110 of P01857 form a hinge region between CH1 and CH2 domain. Positions 111 to 223 of P01857 form the CH2 domain. Positions 224 to 330 of P01857 form the CH3 domain.

Each light chain is comprised of a light chain variable region (abbreviated herein as VL, VL region, or VL domain) and a light chain constant region. The light chain constant region is comprised of one domain, CL.

Unless otherwise specified, amino acid residue positions in the variable regions, including the position of amino acid sequences, substitutions, deletions and insertions as described herein, are numbered according to Kabat numbering (Kabat 1991).

Unless otherwise specified, amino acid residue positions in the constant regions, including the position of amino acid sequences, substitutions, deletions and insertions as described herein, are numbered according to EU numbering (Edelman 1969).

The VH and VL regions can be further subdivided into regions of hypervariability, termed Complementarity Determining Regions (CDR), interspersed with regions that are more conserved, termed framework regions (FW). Each VH and VL is composed of three CDRs and four FWs, arranged from amino-terminus to carboxy-terminus in the following order: FW1, CDR1, FW2, CDR2, FW3, CDR3, FW4. Framework regions can be designated according to their respective VH and VL regions. Thus, e.g., VH-FW1 would refer to the first framework region of VH. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

As used herein, the term “heavy chain region” means a polypeptide that comprises at least one of the CH1, CH2, CH3, CH4 and VH domains. In one embodiment the heavy chain region comprises a heavy chain constant region that comprises CH1, CH2, and CH3, optionally further comprising CH4. In one embodiment the heavy chain region comprises a heavy chain constant region and a heavy chain variable region.

As used herein, the term “light chain region” means a polypeptide that comprises at least one of the CL and VL domains.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site (also referred to as a binding site) within the variable region of the immunoglobulin molecule.

An antibody can be of any the five major classes of immunoglobulins: IgA, IgD, IgE, IgG, and IgM, or subclasses (isotypes) (e.g. IgG1, IgG2, IgG3, IgG4, IgA1 and IgA2), or allotype (e.g., Gm, e.g., G1m(f, z, a or x), G2m(n), G3m(g, b, or c), Am, Em, and Km(1, 2 or 3)). The different classes of immunoglobulins have different and well known subunit structures and three-dimensional configurations. Antibodies may be derived from any mammal, including, but not limited to, humans, monkeys, pigs, horses, rabbits, dogs, cats, mice, etc., or other animals such as birds (e.g. chickens).

The terms “antigen-binding fragment” refers to a fragment comprising antigenic determining variable regions of an intact antibody. It is known in the art that the antigen binding function of an antibody can be performed by fragments of a full-length antibody. Examples of antibody fragments include, but are not limited to Fab, Fab′, F(ab′)2, Fv fragments, scFvs, linear antibodies, single chain antibodies, and multispecific antibodies formed from antibody fragments.

Thus in one embodiment the antibody molecule used in the present invention may comprise a complete antibody molecule having full length heavy and light chains or a fragment thereof and may be, but are not limited to Fab, modified Fab, Fab′, modified Fab′, F(ab′)2, Fv, single domain antibodies (e.g. VH or VL or VHH), scFv, bi, tri or tetra-valent antibodies, Bis-scFv, diabodies, triabodies, tetrabodies, combinations of the same and epitope-binding fragments of any of the above.

Other antibody molecules specifically contemplated are “oligoclonal” antibodies which are a predetermined mixture of distinct monoclonal antibodies. See, e.g., PCT publication WO 95/20401; U.S. Pat. Nos. 5,789,208 and 6,335,163. Preferably oligoclonal antibodies consist of a predetermined mixture of antibodies against one or more epitopes are generated in a single cell. More preferably oligoclonal antibodies comprise a plurality of heavy chains capable of pairing with a common light chain to generate antibodies with multiple specificities (e.g., PCT publication WO 04/009618). Oligoclonal antibodies are particularly useful when it is desired to target multiple epitopes on a single target molecule. Those skilled in the art will know or can determine what type of antibody or mixture of antibodies is applicable for an intended purpose and desired need.

Antibody molecules that comprise at least two antigen-binding domains, each of which being capable of binding to a different target may be termed “bispecific antibody molecules”. In one embodiment the antibody molecule is a bispecific antibody molecule.

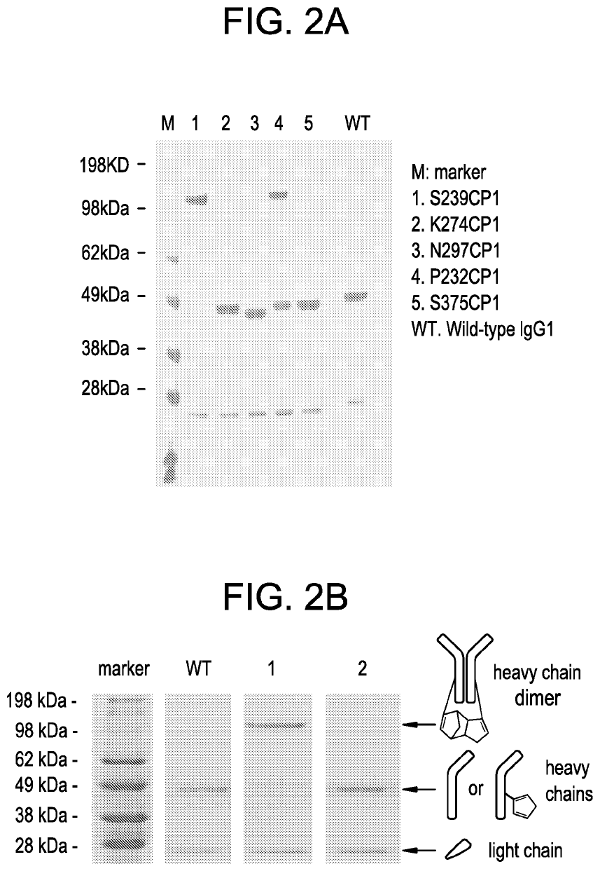

Bispecific antibody molecules may be provided in any suitable format. Suitable formats for a bispecific antibody molecule described herein, and methods for producing the same, are described in Kontermann 2012 and Kontermann 2015, both of which are herein incorporated by reference in their entirety. See in particular FIG. 2 of Kontermann 2012.

Bispecific antibody molecules can also be generated from existing antibodies by chemical conjugation. For example, two IgG molecules or two Fab′ fragments can be coupled using homo- or hetero-bifunctional coupling reagents, e.g. as described in Graziano 2004.

In some embodiments, the bispecific antibody molecules may be an immunoglobulin G-like (IgG-like) bispecific antibody molecule. IgG-like bispecific antibody molecule may comprise an Fv region, Fab region or sVD specific for one antigen, an Fv/Fab/sVD specific for another antigen, and an Fc region. IgG-like bispecific antibody molecules may be either homodimeric (symmetrical) or heterodimeric (asymmetrical).

Homodimeric IgG-like bispecific antibody molecules generally contain an antigen-binding domain that is fused to the N- or C-terminus of the heavy of light chain of an IgG molecule, e.g. in the form of a scFv fragment or a variable single domain. A characteristic property of these homodimeric IgG-like bispecific antibody molecules is that they contain a two identical heavy chains. Furthermore, homodimeric IgG-like bispecific antibody molecules are typically bivalent for each epitope. Valency as used herein refers to the number of antigen-binding regions in the antibody molecule that are able to bind a single epitope. A monoclonal monospecific IgG antibody molecule is bivalent for a single epitope—it contains two antigen-binding domains, each of which are able to bind an epitope on a single target molecule. A homodimeric IgG-like bispecific antibody molecule is bivalent for each epitope—it typically contains four antigen-binding domains, two of which are able to bind a first epitope on a target molecule and two of which are able to bind to a second epitope on a target molecule.

Examples of homodimeric IgG-like bispecific antibody molecules include DVD-IgG, IgG-scFv, scFv-IgG, scFv₄-Ig, IgG-scFab, scFab-IgG, IgG-sVD, sVD-IgG, 2 in 1-IgG, mAb², tandemab common LC. These can be formed by methods known in the art, for example chemical crosslinking, somatic hybridisation or the redox method.

In one embodiment the homodimeric antibody molecule comprises a first and second heavy chain region where the inter-chain disulfides are removed. The inter-chain disulfides may be located at positions 226 and 229 of the first and second heavy chain regions in the antibody molecule, wherein the amino acid residue positions are numbered according to EU numbering. In one embodiment the antibody molecule comprises a first and second heavy chain region, wherein one of the first and second heavy chain regions has a C226 and C229 modification, e.g. a C226V and C229V modification, wherein the amino acid residue positions are numbered according to EU numbering.

Heterodimeric IgG-like bispecific antibody molecules, in contrast, are typically monovalent for each target. As described in, for example, Klein 2012, the concept of monovalent bispecific IgG is thought to have a unique therapeutic niche in that they (i) do not cause receptor homodimerization, (ii) potentially have reduced toxicity on non-target tissues due to loss of avidity for each antigen, and (iii) have better selectivity when both antigens are either selectively restricted or abundantly expressed on target cells. Thus, in some embodiments, the antibody molecule is a heterodimeric IgG-like bispecific antibody molecule.

Heterodimeric IgG-like bispecific antibody molecules involve heterodimerization of two distinct heavy chain and correct pairing of the cognate light chain and heavy chain. Heterodimerization of the heavy chains can be addressed by several techniques, such as knobs-into-holes, electrostatic steering of CH3, CH3 strand exchanged engineered domains and leucine zippers. The pairing of the correct light and heavy chain can be ensured by using one of these heavy chain heterodimerization techniques along with the use of a common light chain, domain cross-over between CH1 and CL, coupling of the heavy and light chains with a linker, in vitro assembly of heavy chain-light chain dimers from two separate monoclonals, interface engineering of an entire Fab domain, or disulfide engineering of the CH1/CL interface.

Examples of heterodimeric IgG-like bispecific antibody molecules include DuetMab, kih IgG, kih IgG common LC, CrossMab, kih IgG-scFab, mAb-Fv, charge pairs and SEED-body. A particular exemplified format of heterodimeric IgG-like bispecific antibody molecules is referred to as “DuetMab”. DuetMab antibody molecules uses KIH technology for heterodimerization of 2 distinct heavy chains and increases the efficacy of cognate heavy and light chain pairing by replacing the native disulphide bond in one of the CH1-CL interfaces with an engineered disulphide bond. Disclosure related to DuetMab can found e.g., in U.S. Pat. No. 9,527,927 and Mazor 2015, which are herein incorporated by reference in their entirety.

In one embodiment the antibody molecule comprise one or more modifications in one or more of the CH1, CH2 and CH3 domains that promotes formation of a heterodimeric antibody molecule. For example, the bispecific antibody molecule described above may additionally comprise one or more modifications in one or more of the CH1, CH2 and CH3 domains that destabilise the formation of a homodimeric antibody molecule and/or promotes formation of a heterodimeric antibody molecule. For example, the antibody molecule may comprise one or more modifications destabilize the homodimer Fc interface.

An example of a modification that destabilizes the homodimer Fc interface is a modification at position 405 of one heavy chain and position 409 in the other heavy chain. This modification is described in more detail in Labrijn et al. 2013. In one embodiment the antibody molecule comprises a first and second heavy chain region, wherein one of the first and second heavy chains regions has a F405 modification, e.g. a F405L modification, and the other heavy chain region has a K409 modification, e.g. a K409R modification, wherein the amino acid residue positions are numbered according to EU numbering.

Other modifications contemplated include a Knobs into Holes (KiH) strategy based on single amino acid substitutions in the CH3 domains that promote heavy chain heterodimerization is described in Ridgway 1996. The knob variant heavy chain CH3 has a small amino acid has been replaced with a larger one, and the hole variant has a large amino acid has replaced with a smaller one. Additional modifications may also introduced to stabilise the association between the heavy chains.

Other binding molecules specifically contemplated for use in the present disclosure are small, engineered protein domains such as scaffold (see for example, U.S. Patent Publication Nos. 2003/0082630 and 2003/0157561). Scaffolds are based upon known naturally-occurring, non-antibody domain families, specifically protein extracellular domains, which typically of small size (˜100 to ˜300 AA) and containing a highly structured core associated with variable domains of high conformational tolerance allowing insertions, deletions or other substitutions. These variable domains can create a putative binding interface for any targeted protein. In general, the design of a generic protein scaffold consists of two major steps: (i) selection of a suitable core protein with desired features and (ii) generation of complex combinatorial libraries by mutagenizing a portion or all of the domains accepting high structural variability, display of these libraries in an appropriate format (i.e., phage, ribosome, bacterial, or yeast) and screening of the library for mutagenized scaffold having the desired binding characteristics (e.g. target specificity and/or affinity). The structure of the parental scaffolds can be highly diverse and include highly structured protein domains including but not limited to, FnIII domains (e.g., AdNectins, see, e.g., Parker 2005, US2008/00139791, and WO 2005/056764, TN3, see e.g., WO2009/058379 and WO2011/130324); Z domains of protein A (Affibody, see, e.g., Wikman 2004 and EP1641818A1); domain A from LDL receptor (Avimers, see, e.g., Silverman 2005 and Braddock 2007); Ankyrin repeat domains (DARPins, Binz 2003, Kohl 2003 and WO02/20565); C-type lectin domains (Tetranectins, see, e.g., WO02/48189). If desired two or more such engineered scaffold domains can be linked together, to form a multivalent binding protein. The individual domains can target a single type of protein or several, depending upon the use/disease indication.

Virtually any molecule (or a portion thereof, e.g., subunits, domains, motifs or a epitope) may be targeted by and/or incorporated into a binding member including, but not limited to, integral membrane proteins including ion channels, ion pumps, G-protein coupled receptors, structural proteins; adhesion proteins such as integrins; transporters; proteins involved in signal transduction and lipid-anchored proteins including G proteins, enzymes such as kinases including membrane-anchored kinases, membrane-bound enzymes, proteases, lipases, phosphatases, fatty acid synthetases, digestive enzymes such as pepsin, trypsin, and chymotrypsin, lysozyme, polymerases; receptors such as hormone receptors, lymphokine receptors, monokine receptors, growth factor receptors, cytokine receptors; cytokines; and more.

In some aspects a binding molecule employed in the present disclosure targets and/or incorporates all or a portion (e.g., subunits, domains, motifs or a epitope) of a growth factor, a cytokine, a cytokine-related protein, a growth factor, a receptor ligand or a receptor selected from among, for example, BMP1, BMP2, BMP3B (GDF10), BMP4, BMP6, BMP8, CSF1 (M-CSF), CSF2 (GM-CSF), CSF3 (G-CSF), EPO, FGF1 (αFGF), FGF2 (pFGF), FGF3 (int-2), FGF4 (HST), FGF5, FGF6 (HST-2), FGF7 (KGF), FGF9, FGF10, FGF11, FGF12, FGF12B, FGF14, FGF16, FGF17, FGF19, FGF20, FGF21, FGF23, FGFR, FGFR1, FGFR2, FGFR3, FGFR4, FGFRL1, FGFR6, IGF1, IGF2, IGF1R, IGF2R, IFNA1, IFNA2, IFNA4, IFNA5, IFNA6, IFNA7, IFNAR1, IFNAR2, IFNB1, IFNG, IFNW1, FIL1, FIL1 (EPSILON), FIL1 (ZETA), IL1A, IL1B, IL2, IL3, IL4, IL5, IL6, IL7, IL8, IL9, IL10, IL11, IL12A, IL12B, IL13, IL14, IL15, IL16, IL17, IL17B, IL18, IL19, IL20, IL22, IL23, IL24, IL25, IL26, IL27, IL28A, IL28B, IL29, IL30, IL2RA, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL11RA, IL12RB1, IL12RB2, IL13RA1, IL13RA2, IL15RA, IL17R, IL17RA, IL17RB, IL17RC, IL17RD, IL18R1, IL20RA, IL20RB, IL21R, IL22R, IL22RA1, IL23R, IL27RA, IL28RA, PDGFA, PDGFB, PDGFRA, PDGFRB, TGFA, TGFB1, TGFB2, TGFB3, TGFBR1, TGFBR2, TGFBR3, ACVRL1, GFRA1, LTA (TNF-beta), LTB, TNF (TNF-alpha), TNFSF4 (OX40 ligand), TNFSF5 (CD40 ligand), TNFSF6 (FasL), TNFSF7 (CD27 ligand), TNFSF8 (CD30 ligand), TNFSF9 (4-1BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, TNFRSF1A, TNFRSF1B, TNFRSF10A (Trail-receptor), TNFRSF10B (Trail-receptor 2), TNFRSF10C (Trail-receptor 3), TNFRSF10D (Trail-receptor 4), FIGF (VEGFD), VEGF, VEGFB, VEGFC, KDR, FLT1, FLT4, NRP1, IL1HY1, IL1RAP, IL1RAPL1, IL1RAPL2, IL1RN, IL6ST, IL18BP, IL18RAP, IL22RA2, AIF1, HGF, LEP (leptin), PTN, ALK and THPO.

In some aspects a binding molecule employed in the present disclosure targets and/or incorporates all or a portion (e.g., subunits, domains, motifs or a epitope) of a chemokine, a chemokine receptor, or a chemokine-related protein selected from among, for example, CCL1(1-309), CCL2 (MCP-1/MCAF), CCL3 (MIP-1a), CCL4 (MIP-1b), CCL5 (RANTES), CCL7 (MCP-3), CCL8 (mcp-2), CCL11 (eotaxin), CCL13 (MCP-4), CCL15 (MIP-1d), CCL16 (HCC-4), CCL17 (TARC), CCL18 (PARC), CCL19 (MIP-3b), CCL20 (MIP-3a), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCL1 (GRO1), CXCL2 (GRO2), CXCL3 (GRO3), CXCL5 (ENA-78), CXCL6 (GCP-2), CXCL9 (MIG), CXCL10 (IP 10), CXCL11 (I-TAC), CXCL12 (SDF1), CXCL13, CXCL14, CXCL16, PF4 (CXCL4), PPBP (CXCL7), CX3CL1 (SCYD1), SCYE1, XCL1 (lymphotactin), XCL2 (SCM-1b), BLR1 (MDR15), CCBP2 (D6/JAB61), CCR1 (CKR1/HM145), CCR2 (mcp-1RB/RA), CCR3 (CKR3/CMKBR3), CCR4, CCR5 (CMKBR5/ChemR13), CCR6 (CMKBR6/CKR-L3/STRL22/DRY6), CCR7 (CKR7/EB11), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Ra), IL8RB (IL8Rb), LTB4R (GPR16), TCP10, CKLFSF2, CKLFSF3, CKLFSF4, CKLFSF5, CKLFSF6, CKLFSF7, CKLFSF8, BDNF, C5R1, CSF3, GRCC10 (C10), EPO, FY (DARC), GDF5, HIF1A, IL8, PRL, RGS3, RGS13, SDF2, SLIT2, TLR2, TLR4, TREM1, TREM2, and VHL.

In some aspects a binding molecule employed in the present disclosure targets and/or incorporates all or a portion (e.g., subunits, domains, motifs or a epitope) of a protein selected from among, for example renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VII, factor VIIIC, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor; epidermal growth factor (EGF); insulin-like growth factor binding proteins; CD proteins such as CD2, CD3, CD4, CD 8, CD11a, CD14, CD18, CD19, CD20, CD22, CD23, CD25, CD33, CD34, CD40, CD40L, CD52, CD63, CD64, CD80 and CD147; erythropoietin; osteoinductive factors; immunotoxins; superoxide dismutase; T-cell receptors; surface membrane proteins; decay accelerating factor; viral antigen such as, for example, a portion of the AIDS envelope, e.g., gp120; transport proteins; homing receptors; addressins; regulatory proteins; cell adhesion molecules such as LFA-1, Mac 1, p150.95, VLA-4, ICAM-1, ICAM-3 and VCAM, a4/p7 integrin, and (Xv/p3 integrin including either a or subunits thereof, integrin alpha subunits such as CD49a, CD49b, CD49c, CD49d, CD49e, CD49f, alpha7, alpha8, alpha9, alphaD, CD11a, CD11b, CD51, CD11c, CD41, alphaIIb, alphaIELb; integrin beta subunits such as, CD29, CD 18, CD61, CD104, beta5, beta6, beta7 and beta8; Integrin subunit combinations including but not limited to, αVβ3, αVβ5 and α4β7; a member of an apoptosis pathway; IgE; blood group antigens; flk2/flt3 receptor; obesity (OB) receptor; mpl receptor; CTLA-4; protein C; an Eph receptor such as EphA2, EphA4, EphB2, etc.; a Human Leukocyte Antigen (HLA) such as HLA-DR; complement proteins such as complement receptor CR1, C1Rq and other complement factors such as C3, and C5; a glycoprotein receptor such as GpIbα, GPIIb/IIIa and CD200.

Also contemplated are binding molecules that specifically bind and/or comprises cancer antigens including, but not limited to, ALK receptor (pleiotrophin receptor), pleiotrophin, KS 1/4 pan-carcinoma antigen; ovarian carcinoma antigen (CA125); prostatic acid phosphate; prostate specific antigen (PSA); melanoma-associated antigen p97; melanoma antigen gp75; high molecular weight melanoma antigen (HMW-MAA); prostate specific membrane antigen; carcinoembryonic antigen (CEA); polymorphic epithelial mucin antigen; human milk fat globule antigen; colorectal tumor-associated antigens such as: CEA, TAG-72, CO17-1A, GICA 19-9, CTA-1 and LEA; Burkitt's lymphoma antigen-38.13; CD19; human B-lymphoma antigen-CD20; CD33; melanoma specific antigens such as ganglioside GD2, ganglioside GD3, ganglioside GM2 and ganglioside GM3; tumor-specific transplantation type cell-surface antigen (TSTA); virally-induced tumor antigens including T-antigen, DNA tumor viruses and Envelope antigens of RNA tumor viruses; oncofetal antigen-alpha-fetoprotein such as CEA of colon, 5T4 oncofetal trophoblast glycoprotein and bladder tumor oncofetal antigen; differentiation antigen such as human lung carcinoma antigens L6 and L20; antigens of fibrosarcoma; human leukemia T cell antigen-Gp37; neoglycoprotein; sphingolipids; breast cancer antigens such as EGFR (Epidermal growth factor receptor); NY-BR-16, NY-BR-16, HER2 antigen (p185HER2), and HER3; polymorphic epithelial mucin (PEM); malignant human lymphocyte antigen-APO-1; differentiation antigen such as I antigen found in fetal erythrocytes; primary endoderm I antigen found in adult erythrocytes; preimplantation embryos; I(Ma) found in gastric adenocarcinomas; M18, M39 found in breast epithelium; SSEA-1 found in myeloid cells; VEP8; VEP9; Myl; VIM-D5; D156-22 found in colorectal cancer; TRA-1-85 (blood group H); SCP-1 found in testis and ovarian cancer; C14 found in colonic adenocarcinoma; F3 found in lung adenocarcinoma; AH6 found in gastric cancer; Y hapten; Ley found in embryonal carcinoma cells; TL5 (blood group A); EGF receptor found in A431 cells; E1 series (blood group B) found in pancreatic cancer; FC10.2 found in embryonal carcinoma cells; gastric adenocarcinoma antigen; CO-514 (blood group Lea) found in Adenocarcinoma; NS-10 found in adenocarcinomas; CO-43 (blood group Leb); G49 found in EGF receptor of A431 cells; MH2 (blood group ALeb/Ley) found in colonic adenocarcinoma; 19.9 found in colon cancer; gastric cancer mucins; T5A7 found in myeloid cells; R²⁴ found in melanoma; 4.2, GD3, D1.1, OFA-1, GM2, OFA-2, GD2, and M1:22:25:8 found in embryonal carcinoma cells and SSEA-3 and SSEA-4 found in 4 to 8-cell stage embryos; Cutaneous Tcell Lymphoma antigen; MART-1 antigen; Sialy Tn (STn) antigen; Colon cancer antigen NY-CO-45; Lung cancer antigen NY-LU-12 variant A; Adenocarcinoma antigen ART1; Paraneoplastic associated brain-testis-cancer antigen (onconeuronal antigen MA2; paraneoplastic neuronal antigen); Neuro-oncological ventral antigen 2 (NOVA2); Hepatocellular carcinoma antigen gene 520; TUMOR-ASSOCIATED ANTIGEN CO-029; Tumor-associated antigens MAGE-C1 (cancer/testis antigen CT7), MAGE-B1 (MAGE-XP antigen), MAGE-B2 (DAM6), MAGE-2, MAGE-4a, MAGE-4b and MAGE-X2; Cancer-Testis Antigen (NY-EOS-1) and fragments of any of the above-listed polypeptides.

In one embodiment the polypeptide employed is recombinant. A “recombinant” polypeptide or protein refers to a polypeptide or protein produced via recombinant DNA technology. Recombinantly produced polypeptides and proteins expressed in engineered host cells are considered isolated for the purpose of this disclosure, as are native or recombinant polypeptides which have been separated, fractionated, or partially or substantially purified by any suitable technique. The polypeptides disclosed herein can be recombinantly produced using methods known in the art. Alternatively, the proteins and peptides disclosed herein can be chemically synthesized.

In one embodiment the first polypeptide is or comprises a binding molecule, such as an antibody molecule and the second polypeptide is or comprises a synthetic IgG-binding domain. Examples of an IgG-binding domains include protein Z (also known as a “ZZ-tag”) described in Nilsson 1987. In one embodiment the first polypeptide is or comprises an antibody molecule and the second polypeptide is or comprises protein Z.

The first and second polypeptides may be first and second component polypeptides of a split protein, such that the first and second component polypeptides form a split protein upon dimerization. Examples of split proteins include split chimeric antigen receptor (CAR; e.g. as described in Wu 2015), split kinases (e.g. as described in Camacho-Soto 2014), split transcription factors (e.g. as described in Taylor 2010) and split caspases (e.g. as described in Chelur 2007). Typically, component polypeptides of split proteins contain homo- or heter-dimerization domains that conditionally interact upon binding of a small molecule. In one embodiment the split protein has increased activity upon dimerization of the first and second component polypeptide. These first and second component polypeptides can include a CP moiety such that upon dimerization, e.g. by a small molecule, the first and second polypeptides become covalently linked and therefore locked in the dimerized state.

In one embodiment the conjugate of the first and second component polypeptides form a split chimeric antigen receptor that is capable of activating a T-cell and wherein dimerization of the first and second component polypeptides results in increased activation of the T-cell. Activating a T-cell in this context may mean being able to stimulate proliferation of the T-cells. The split chimeric antigen receptor may be present in a T-cell (CAR-T) and the CAR-T cell may be modified ex vivo to include the CP moiety on the first and/or second component polypeptides.

In one embodiment the conjugate of the first and second component polypeptides form a split caspase that is capable of inducing caspase activity and wherein dimerization of the first and second component polypeptides results in increased caspase activity. Caspase activity in this context may mean the likelihood of the caspase inducing cell death. The split caspase may be present in a cell, e.g. a T-cell, and the cell may be modified ex vivo to include the CP moiety on the first and/or second component polypeptides.

In one embodiment the conjugate of the first and second component polypeptides form a split transcription factor that is capable of transcriptional activity and wherein dimerization of the first and second component polypeptides results in increased transcriptional activity. Transcriptional activity in this context may mean being able to activate or repress transcription. The split transcription factor receptor may be present in a cell and the cell may be modified ex vivo to include the CP moiety on the first and/or second component polypeptides.

In one embodiment an antibody molecule comprises the conjugate of the first and second polypeptides.

As described above, antibody subunits (e.g. heavy chains and light chains) in an antibody molecule are typically interconnected by inter-chain disulfide bonds (e.g. between the two heavy chains of an antibody and between the heavy chain and light chain of an antibody). The present inventors found that the CPD moiety can be used in addition to or instead of the disulfide bonds to covalently link antibody subunits to generate homodimeric and/or heterodimeric antibody molecules with unique assembly and stability properties. Once formed, the Diels-Alder adduct is not susceptible to reduction, but can be reversed by heat (>70° C.) or application of an electric field. These stability properties are different than natural disulfides, which can be disrupted by reduction and/or enzymatic activity. Furthermore, the self-reaction can be used in combination with other mutations to manipulate protein structure and function in the desired manner. Additionally, the CPD moiety can be included without altering naturally occurring disulfides.

The CP moieties that form the CPD moiety may be located on the heavy chain and/or light chain in an antibody molecule. In one embodiment the antibody molecule comprises a conjugate of a first polypeptide and a second polypeptide, wherein the first polypeptide comprises a first heavy chain region and the second polypeptide comprises a second heavy chain region, and wherein the link between the first heavy chain region and second heavy chain region comprises the CPD moiety. The antibody molecule may be a bispecific antibody molecule.

In one embodiment the antibody molecule comprises a conjugate of a first polypeptide and a second polypeptide, wherein the first polypeptide comprises a first heavy chain and the second polypeptide comprises a first light chain region, wherein the link between the first heavy chain region and first light chain region comprises the CPD moiety. The antibody molecule may be a scFV. The antibody molecule may further comprise an Fc region, for example the antibody molecule may be an scFv-Fc fusion.

The CP moieties may be located anywhere on the heavy and/or light chain in an antibody molecule provided that when the antibody molecule is fully folded and assembled the CP moieties are considered likely able to interact and enable Diels-Alder (DA) dimerization. As described elsewhere, it is possible to determine whether CP moieties are likely able to interact and enable DA dimerization by making use of the molecular structure, e.g. crystal structure, that comprises the first polypeptide (e.g. the first heavy chain region) and second polypeptide (e.g. the second heavy chain region or light chain region). As described in the examples, for antibody molecules this can be done using the crystal structure from PDB entry 1FC1 (version 1.2) and the PyMol software.

In one embodiment the link between the first heavy chain region and second heavy chain region comprising the CPD moiety is located between amino acids in one or more of the CH1, CH2, CH3 and hinge regions. In one embodiment the link between the first heavy chain region and second heavy chain region comprising the CPD moiety is located in the CH3 region. In one embodiment the link between the first heavy chain region and second heavy chain region comprising the CPD moiety is located between amino acids in the hinge region or CH2 domain of the first and second heavy chain regions.

In one embodiment the link between the first heavy chain region and second heavy chain region comprising the CPD moiety is located at any one of positions 227, 228, 229, 230, 231, 232, 233, 234, 235, 236, 237, 238, 239, 240 and 241 of the first and second heavy chain regions, wherein the amino acid residue positions are numbered according to EU numbering. In one embodiment the link between the first heavy chain region and second heavy chain region comprising the CPD moiety is located at any one of positions 232, 233, 234, 235, 236, 237 and 239 of the first and second heavy chain regions, wherein the amino acid residue positions are numbered according to EU numbering. In one embodiment the link between the first heavy chain region and second heavy chain region comprising the CPD moiety is located at positions 232 or 239 of the first and second heavy chain regions, wherein amino acid residue positions are numbered according to EU numbering. In one embodiment the link between the first heavy chain region and second heavy chain region comprising the CPD moiety is located at position 239 of the first and second heavy chain regions, wherein the amino acid residue position is numbered according to EU numbering. In one embodiment the link between the first heavy chain region and second heavy chain region comprising the CPD moiety is located at position 232 of the first and second heavy chain regions, wherein the amino acid residue position is numbered according to EU numbering.

As mentioned above, the CPD moiety can be used instead of the disulfide bonds to covalently link antibody subunits to generate homodimeric and/or heterodimeric antibody molecules with unique assembly and stability properties. In one embodiment the antibody molecule comprises a first and second heavy chain region where the inter-chain disulfides are removed. The inter-chain disulfides may normally be located at positions 226 and 229 of the first and second heavy chain regions in the antibody molecule, e.g. an IgG1, wherein the amino acid residue positions are numbered according to EU numbering. In one embodiment the antibody molecule comprises a first and second heavy chain region, wherein one or both of the first and second heavy chain regions has a residue other than cysteine at positions 226 and 229. In one embodiment the antibody molecule is a homodimeric antibody molecule, wherein the first and second heavy chain regions have a valine (V) residue at positions 226 and 229, wherein the amino acid residue positions are numbered according to EU numbering.

The inter-chain disulfides in a heterodimeric antibody molecule may be replaced by a charged pair. As described in Brinkmann and Kontermann, 2017, the charged pairs have been used to favour heterodimeric assembly of bispecific antibody molecules. In one embodiment the heterodimeric antibody molecule comprises a first and second heavy chain region, wherein one of the first and second heavy chain regions comprises a positively charged amino acid (lysine (K), histidine (H) or arginine (R)) at positions 226 and/or 229, and the other heavy chain region comprises a negatively charged amino acid (aspartic acid (D) or glutamic acid (E)) at positions 226 and/or 229. In one embodiment one of the first and second heavy chain regions comprises a glutamic acid (E) residue at positions 226 and 229, and the other heavy chain comprises an arginine (R) residue at positions 226 and 229, wherein the amino acid residue positions are numbered according to EU numbering.

The heterodimeric antibody molecules may additionally comprise one or more modifications that promote the formation of a heterodimeric antibody molecule as described above. For example, the heterodimeric antibody molecules may comprise one or more modifications that destabilise the formation of a homodimeric antibody molecule. In one embodiment one of the first and second heavy chain regions comprises a glutamic acid (E) residue at positions 226 and 229 and a leucine (L) residue at position 405, and the other heavy chain comprises an arginine (R) residue at positions 226, 229 and 409, wherein the amino acid residue positions are numbered according to EU numbering.

In one embodiment the link between the first heavy chain region and second light chain region comprising the CPD moiety is located between an amino acid in the VH domain of the first heavy chain region and an amino acid in the VL region of the second light chain region. In one embodiment the link between the first heavy chain region and second light chain region comprising the CPD moiety is located at position 39 in the heavy chain variable region and position 42 in the light chain variable region, wherein the amino acid residue positions are numbered according to Kabat numbering.

Methods

In one embodiment, the method of conjugating a first polypeptide and a second polypeptide comprises expressing one or more nucleic acids encoding the first and second polypeptide in one or more host cells, adding a non-natural amino acid comprising a CP moiety to the one or more host cells under conditions sufficient to incorporate the CP moiety into the first and second polypeptide, culturing the one or more host cells under conditions that allow a Diel-Alder reaction to occur between the CP moieties to produce a conjugate between the first and second polypeptide, and optionally isolating and/or purifying the conjugate.

One or more isolated nucleic acid molecules may be used to express the first and second polypeptide described herein. The nucleic acid will generally be provided in the form of a recombinant vector for expression. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g. phage, or phagemid, as appropriate.

In one embodiment the first and second polypeptides are expressed from separate nucleic acids, e.g. separate vectors. That is, the method comprises expressing a first nucleic acid encoding the first polypeptide in a host cell and expressing a second nucleic acid encoding a second polypeptide in the same host cell or a different host cell. In another embodiment the first and second polypeptides are expressed from the same nucleic acid, e.g. the same vector.

Techniques for the introduction of nucleic acid or vectors into host cells are well established in the art and any suitable technique may be employed. A range of host cells suitable for the production of recombinant polypeptides are known in the art, and include bacterial, yeast, insect or mammalian host cells. A preferred host cell is a mammalian cell, such as a CHO, NSO, or HEK cell, for example a HEK293 cell.

Methods for incorporating a non-natural amino acid into the polypeptide sequence are described in the Examples and in WO 2018/218093. As described herein the Diel-Alder reaction may occur during culture of the host cells when the CP moieties are located at amino acid positions in the first and second polypeptides that are at a sufficient proximity in the assembled protein structure. The assembled protein structure may be a heterodimer between the first and second polypeptides. The cells may be cultured for at least 1, 3, 7, or 11 days. Methods for culturing host cells are well-known in the art. The first and second polypeptides may be secreted by the host cells into the cell culture fluid.

In one embodiment the method comprises expressing the one or more nucleic acids encoding the first and second polypeptide in the same host cell, e.g. a mammalian host cell. In such a case, expression of both the first and second polypeptide occurs in the same cell. Such a method may be referred to herein as a “cotransfect” method. The Diel-Alder reaction between the first and second polypeptides may occur in the host cell or outside the host cell, e.g. in the cell culture fluid.

In one embodiment the method comprises expressing a first nucleic acid encoding the first polypeptide in a first host cell and expressing a second nucleic acid encoding the second polypeptide in a second host cell. In such a case, expression of the first polypeptide occurs in the first cell and expression of the second polypeptide occurs in the second cell. Such a method may be referred to herein as a “coculture” method. The Diel-Alder reaction between the first and second polypeptides will occur outside the host cells, e.g. in the cell culture fluid.

The conjugate being produced may be an antibody molecule. The antibody molecule may be a heterodimeric antibody molecule as described herein where the first polypeptide comprises a first heavy chain region and the second polypeptide comprises a second heavy chain region that differs from the first heavy chain region in amino acid sequence. The first and second heavy chain region may contain any of the modifications to promote formation of a heterodimeric antibody molecule described herein, for example the modifications to remove inter-chain disulfide bonds between the heavy chains and/or the modifications to destabilise the formation of a homodimeric antibody molecule. As described in the examples, both the “cotransfect” method and the “coculture” method can be used to efficiently produce bispecific antibody molecules.

For example, where the method comprises producing a heterodimeric antibody molecule using the “coculture” method, the first polypeptide may form a first homodimer made up of two copies of the first polypeptide in the first cell, and the second polypeptide form a second homodimer made up of two copies of the second polypeptide in the second cell. Without wishing to be bound by theory, it is believed that non-covalent interactions between the CH3 domains allow the heavy chains to associate with each other to form the first and second homodimers and that this happens even in the presence of modifications that destabilise the formation of a homodimeric antibody molecule.

Both the first and second homodimer may be secreted into the cell culture fluid where extracellular heavy chain exchange can occur to produce the heterodimeric antibody molecule comprising the first polypeptide and the second polypeptide and the Diel-Alder reaction occurs to produce the conjugate that is covalently linked by the CPD moiety. The modifications to promote formation of the heterodimeric antibody molecules described herein may encourage the heavy chain exchange to produce heterodimeric antibody molecules rather than homodimeric antibody molecules. As demonstrated in the examples, this “coculture” method was used to bispecific antibody molecules where the main product formed was the heterodimeric antibody molecule.

The method may further comprise isolating and/or purifying the conjugate. Techniques for the purification of recombinant polypeptides are well-known in the art and include, for example HPLC, FPLC or affinity chromatography, e.g. using Protein A or Protein L. Where the conjugate being produced is a heterodimeric antibody molecule, the method may further comprise isolating or purifying the heterodimeric antibody molecules from homodimeric antibody molecules produced by the method.

### Other Definitions

Before describing the provided embodiments in detail, it is to be understood that this disclosure is not limited to specific compositions or process steps, and as such can vary. As used in this specification and the appended claims, the singular forms “a”, “an” and “the” include plural referents unless the context clearly dictates otherwise. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects, which can be had by reference to the specification as a whole. Accordingly, the terms defined immediately below are more fully defined by reference to the specification in its entirety. Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

The word “label” when used herein refers to a detectable compound or composition which is conjugated directly or indirectly to an engineered antibody or fragment thereof disclosed herein (e.g., a cysteine engineered antibody or fragment thereof) so as to generate a “labeled” conjugate compound. The label can be detectable by itself (e.g., radioisotope labels or fluorescent labels) or, in the case of an enzymatic label, can catalyze chemical alteration of a substrate compound or composition that is detectable.

The terms “polynucleotide” and “nucleic acid” are used interchangeably herein and refer to polymers of nucleotides of any length, including DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs.

As used herein, the term “vector” refers to a construct, which is capable of delivering, and in some aspects, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, the term “comprising” in context of the present specification should be interpreted as “including”.

“Employed in the present disclosure” as used herein refers to employed in the method disclosed herein, employed in the molecules including intermediates disclosed herein or both, as appropriate to the context of the term used.

It is understood that wherever aspects are described herein with the language “comprising,” otherwise analogous aspects described in terms of “consisting of” and/or “consisting essentially of” are also provided.

Any positive embodiment or combination thereof described herein may be the basis of a negative exclusion i.e. a disclaimer.

In the context of this specification “comprising” is to be interpreted as “including”.

Embodiments of the invention comprising certain features/elements are also intended to extend to alternative embodiments “consisting” or “consisting essentially” of the relevant elements/features.

Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

### Acronyms

ADC Antibody drug conjugate

mAb Monoclonal antibody

MS Mass spectrometry

MS/MS Tandem mass spectrometry

NNAA Non-natural amino acid

RP-HPLC Reverse phase high performance liquid chromatography analysis

SEC Size exclusion chromatography

WT Wild-type

The present invention is further described by way of illustration only in the following examples, which refer to the accompanying Figures, in which:

### EXAMPLES

### Example 1—Materials and Methods

Chemistry Materials and Methods

CpK (also termed CP1 nnAA) was synthesized as described in Example 2 of WO 2018/218093. CpK is represented as 4 in WO 2018/218093.

Bioanalytical Methods

Mass Spectrometry (MS) Analysis of Antibodies and ADCs

Mass spectrometry analysis was performed using an Agilent 6520B Q-TOF mass spectrometer equipped with a RP-HPLC column (ZORBAX 300 Diphenyl RRHD, 1.8 micron, 2.1 mm×50 mm). High-performance liquid chromatography (HPLC) parameters were as follows: flow rate, 0.5 ml/min; mobile phase A was 0.1% (v/v) formic acid in HPLC-grade H₂O, and mobile phase B was 0.1% (v/v) formic acid in acetonitrile. The column was equilibrated in 90% A/10% B, which was also used to desalt the mAb samples, followed by elution in 20% A/80% B. Mass spec data were collected for 100-3000 m/z, positive polarity, a gas temperature of 350° C., a nebulizer pressure of 48 lb/in², and a capillary voltage of 5,000 V. Data were analyzed using vendor-supplied (Agilent v.B.04.00) MassHunter Qualitative Analysis software and peak intensities from deconvoluted spectra were used to derive the relative proportion of species in each sample as previously described (Valliere-Douglass et al. 2012). For deglycosylated mAb analysis, EndoS (5 μL Remove-iT EndoS (1:10 dilution in PBS, 20,000 units/mL, New England BioLabs) was combined with 50 μL sample (1 mg/mL mAb) and 5 μL glyco buffer 1 (New England BioLabs) and followed by incubation for 1 h at 37° C. Reduced samples were prepared by addition of 5 μL Bond-Breaker TCEP solution (0.5 M, Thermo Fisher Scientific) and incubation for 10 min at 37° C.

Size Exclusion Chromatography (SEC)

SEC analysis was performed using an Agilent 1100 Capillary LC system equipped with a triple detector array (Viscotek 301, Viscotek, Houson, Tex.); the wavelength was set to 280 nm, and samples were run on a TSK-GEL G3000SWXL column (Toso Bioscience LLC, Montgomeryville, Pa.) using 100 mM sodium phosphate buffer, pH 6.8 at a flow rate of 1 mL/min. Antibody solutions were typically prepared at 1 mg/mL in phosphate buffer, and 50-100 μL was injected into the instrument for each analysis. Percent monomer was determined using integrated peak areas from the chromatogram.

Reverse Phase High Performance Liquid Chromatography Analysis (RP-HPLC)

For each analysis, 8-12 μg of antibody sample was loaded onto a PLRP-S, 1000 Å column (2.1×50 mm, Agilent) and eluted at 80° C. at a flow rate of 0.5 mL/min with a gradient of 5% B to 100% B over 25 minutes (mobile phase A: 0.1% trifluoroacetic acid in water, and mobile phase B: 0.1% trifluoroacetic acid in acetonitrile).

Generation of Antibody Peptide Fragments and RP-HPLC/MS Analysis

Antibodies were first cleaved by addition of IdeS at a 1:3 ratio (mass/mass) followed by incubation overnight at room temperature. The cleaved Fc portions were purified from enzyme on a 1 mL MabSelect Sure column (GE Healthcare) and eluted with 0.1 M glycine, pH 2.8. Following intact mass analysis of the Fc portions (as above), these proteins were then denatured with 8 M guandidine, reduced and alkylated with 25 mM DTT and 50 mM iodoacetamide, then digested for 4 hours with trypsin after buffer exchange to 2M urea. Digested peptides were desalted using an OASIS HLB desalting plate (Waters). Tandem mass spectrometry analysis of peptide samples was carried out on an Orbitrap Fusion Tribrid (Thermo Fisher Scientific) MS operated in positive polarity and interfaced with a Dionex 3000 nanoRSLC system. Peptides were captured using trap column (2 cm×75 μm ReproSil-Pur 120 C18-AQ, 5 μm size) and separated on an analytical column (20 cm×75 μm ReproSil-Pur 120 C18-AQ, 1.9 μm size) both packed. Reversed-phase solvent gradient consisted of 0.1% formic acid with increasing amount of solvent B (80% acetonitrile in 0.1% formic acid) over a period of 60 minutes. Peptides were separated using linear gradient of solvent B from 5-25% for 26 min, 25-32% for 8 min, 32-45% for 5 min, 45-95% for 3 min and stayed at 95% for 2 min.

Antibody Design

Antibodies were generated to comprise CP1 nnAA (also termed “CpK”) at desired positions by mutation of native amino acid codons to a TAG codon in the gene sequence. The general notation: AXCP1 is used to indicate the amino acid mutation to CP1 where A=native amino acid one-letter code, X=amino acid number, CP1=CP1 nnAA. In some examples, CP1 nnAA was incorporated into antibodies with additional natural amino acid mutations as indicated.

Expression of Antibodies

IgG1 antibody genes with an amber mutation (TAG) at the desired Fc positions were cloned into a proprietary pOE antibody expression vector. The construct was transfected into CHO-G22 by PElImax (1.5 L of G22 cells), along with a plasmid encoding PyIRS double mutant (Y306A/Y384F) and a plasmid containing tandem repeats of the tRNA expression cassette (pORIP 9X tRNA). Four hours post transfection, 3.5% of feed (proprietary) solution was added to cells and the cells were further incubated at 34° C. CpK was added to transfected cells the next day at final concentration of 0.5 mM (from 260 mM stock in 0.2 M NaOH). Cells were fed again on day 3 and day 7 with 7% of feed solution. Cells were spun down and supernatant was harvested on day 11. Antibody was purified from the supernatant using an IgSelect affinity column (GE Health Care Life Science) and eluted with 50 mM glycine, 30 mM NaCl, pH 3.5 buffer, then neutralized with 1 M Tris buffer pH 7.5 and finally dialyzed into PBS, pH 7.2. Antibody concentration was determined by absorbance measurement at 280 nm and expression titers were back-calculated based on final recovered antibody and total volume of cell culture media used. Recovered antibodies were analyzed by sodium docecyl sulfate poly(acrylamide) gel electrophoresis (SDS-PAGE) using standard methods. Antibodies were also analyzed by mass spectrometry (MS), reduced reverse-phase high performance liquid chromatography (rRP-HPLC) and size exclusion chromatography (SEC).

### Example 2—Incorporation of CpK into Antibodies and Antibody Dimerization Through Diels-Alder Crosslinking

1C1 antibody comprised a human IgG1 framework with variable domains binding the EphA2 receptor. Production of the 1C1 antibody is described in US20090304721A1. Natural amino acids in the heavy chain were mutated to CpK to determine dimerization efficiency. Heavy-chain hinge cysteines were not modified, 1C1 antibodies contained natural interchain disulfides. Antibodies were expressed and characterized as described in Example 1.

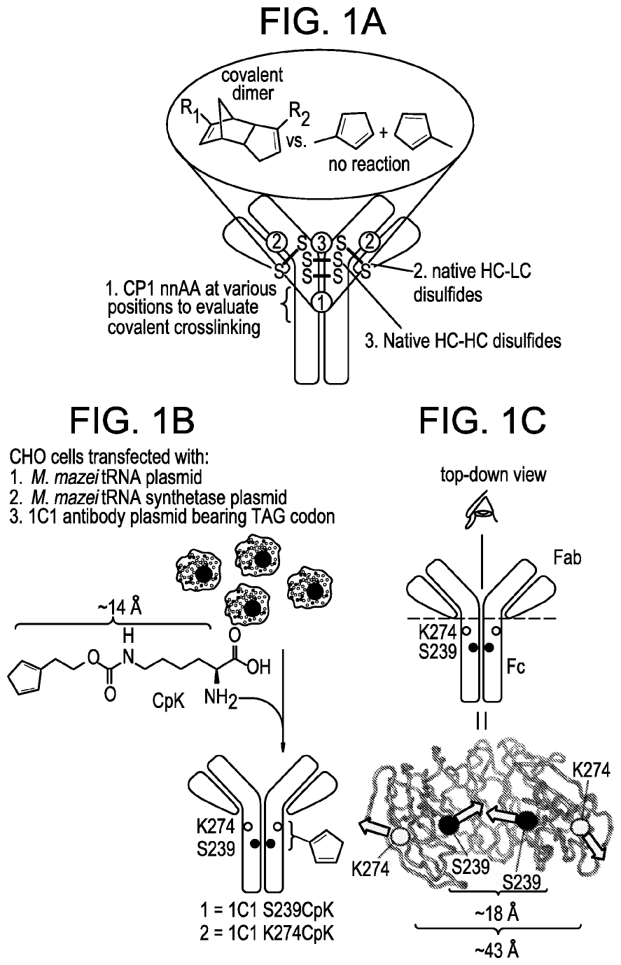

Antibody heavy-chain positions P232, S239, K274, N297 and S375 (EU numbering) were selected for incorporation of CpK following analysis of the known crystal structure of the human IgG1 antibody Fc fragment to estimate distances between amino acid α-carbons and orientation of side chains using PyMOL (Schrodinger LLC) and the crystal structure from PDB entry 1FC1] (Deisenhofer 1981). For example, in the fully folded and assembled antibody molecule, heavy-chain amino acid α-carbons of amino acids at position S239CpK (1) are within ˜18 Å from each other and side chains are likely able to interact and enable Diels-Alder (DA) dimerization, whereas amino acids α-carbons at heavy chain position K274CpK (2) are ˜43 Å and side chains are likely facing away, thus preventing dimerization and enabling reaction with maleimide for bioconjugation (FIG. 1). Bioconjugation at position K274 is described in WO 2018/218093.

A summary of IC1 mAb^{a} properties with CpK incorporated at specified positions is shown in Table 1 below

TABLE 1

| Amino | | Number | Distance | Monomer | Day 10 titer |
|---|---|---|---|---|---|
| acid | Mutation | (EU) | (Å)^{b} | (%)^{c} | (mg/L) |
| P | CP1 | 232 | 7.1 | 96 | 162 |
| S | CP1 | 239 | 17.3 | 94 | 139 |
| K | CP1 | 274 | 43.1 | 91 | 138 |
| N | CP1 | 297 | 31.3 | 71 | 197 |

a. 1C1 mAb is a standard IgG1 antibody with the only mutations being the nnAA at the specified positions.
^{b}Determined by measuring distance between amino acid alpha carbons using crystal structure data.
^{c}Determined by size-exclusion chromatography.

Antibody recovery following purification with protein A was higher than titers reported for expression of azide or cyclopropene ncAAs (approximately 40-80 mg/L) using a similar transient expression system (Oller-Salvia 2018; Vanbrunt 2015). High ncAA titers also correlated with high CHO cell viability (>80% measured at the time of harvest). CpK itself was well tolerated, stable, and biocompatible, as evidenced by high cell viability throughout expression, lack of formation of DA adducts with natural metabolites, or degradation of the diene unit as determined by mass spectrometry (MS) (vida infra). These results demonstrate that the CpK functional group is robust and suitable for applications that demand exposure to complex biological milieu and metabolic processes for extended periods of time.

Antibody analysis by reduced SDS-PAGE (FIG. 2) indicated a high molecular weight species at ˜100 KDa for the S239CpK and P232CpK antibodies that was not present in WT mAb or for the K274CpK, N296CpK or S375CpK. This result suggests that the S239CpK and P232CpK molecules have formed covalent DA adducts through dimerization.

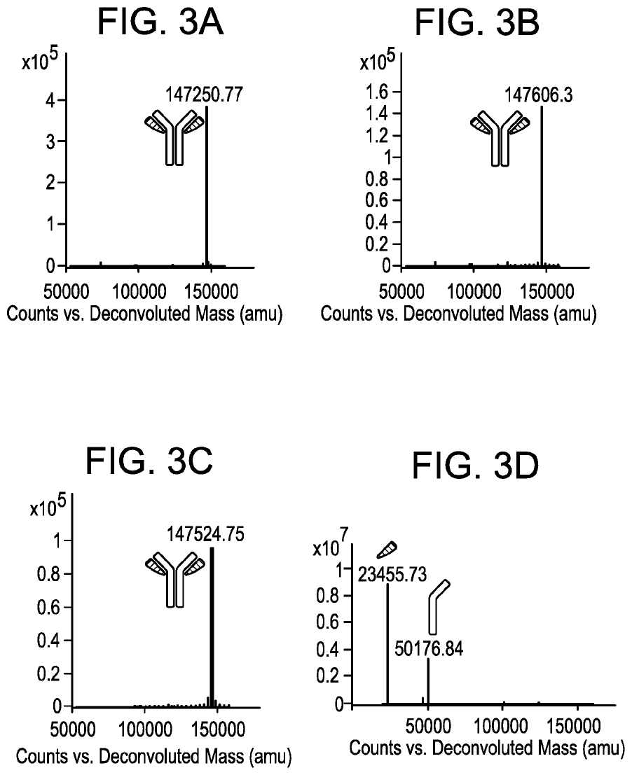

Characterization of intact mAb products by MS showed a single species was obtained for both 1C1 S239CpK and 1C1 K274CpK (FIG. 3B and FIG. C), corresponding to the expected molecular weight after mutation of serine or lysine to CpK. Analysis of the reduced antibody products by MS showed that mAbs 1C1 S239CpK and 1C1 K274CpK were fundamentally different (FIG. 3E and FIG. 3F). Specifically, the wild-type (WT) antibody and 1C1 K274CpK denatured into heavy and light chains after reduction with TCEP (FIG. 3D and FIG. 3F), whereas 1C1 S239CpK denatured into light chains and a higher molecular weight species of ˜100 kDa (FIG. 2 and FIG. 3E). MS analysis confirmed the higher molecular weight species to be 100,708.78 Da, corresponding to heavy-chain dimer containing two CpK amino acids.

FIG. 3G demonstrates that 1C1 P232CpK forms a mixture of both monomeric and dimeric species. Without wishing to be bound by theory, this may indicate that dimerization at position 232 is less efficient that dimerization at position 239.

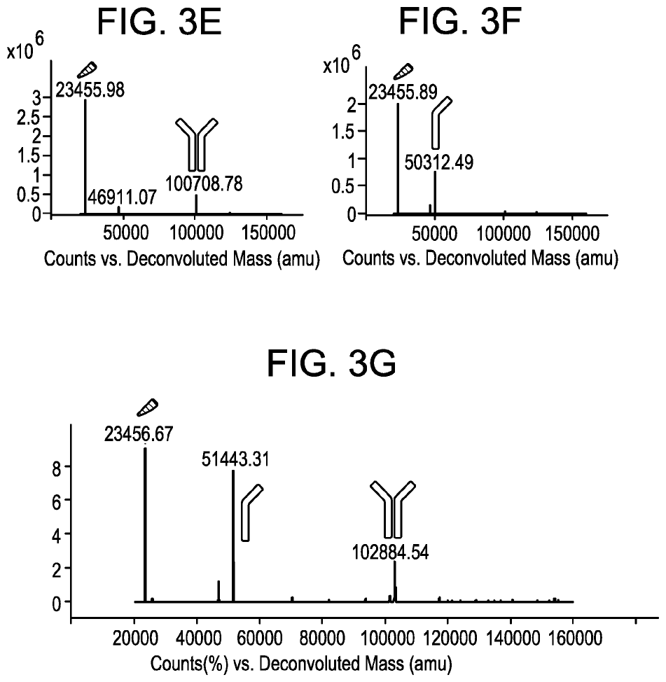

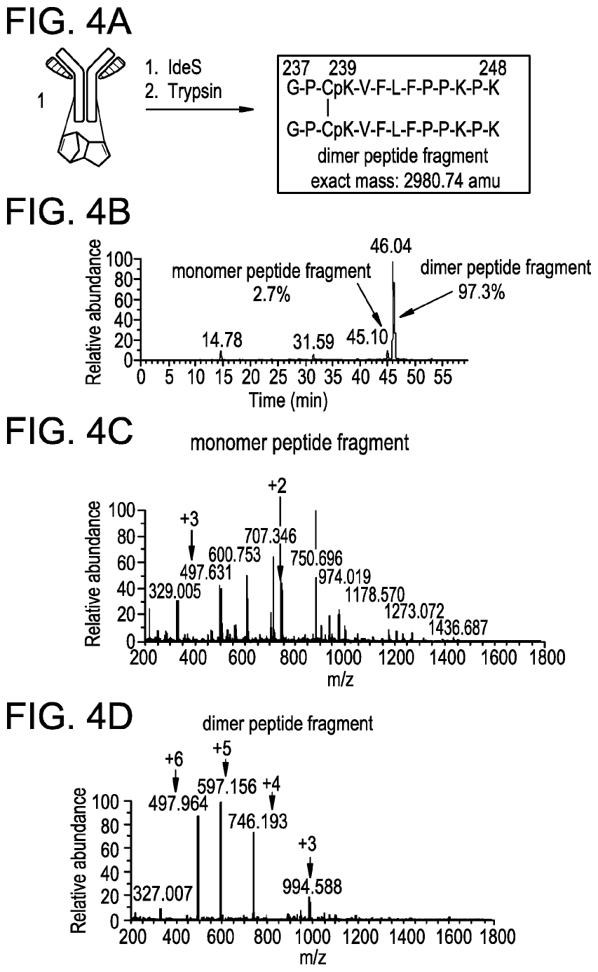

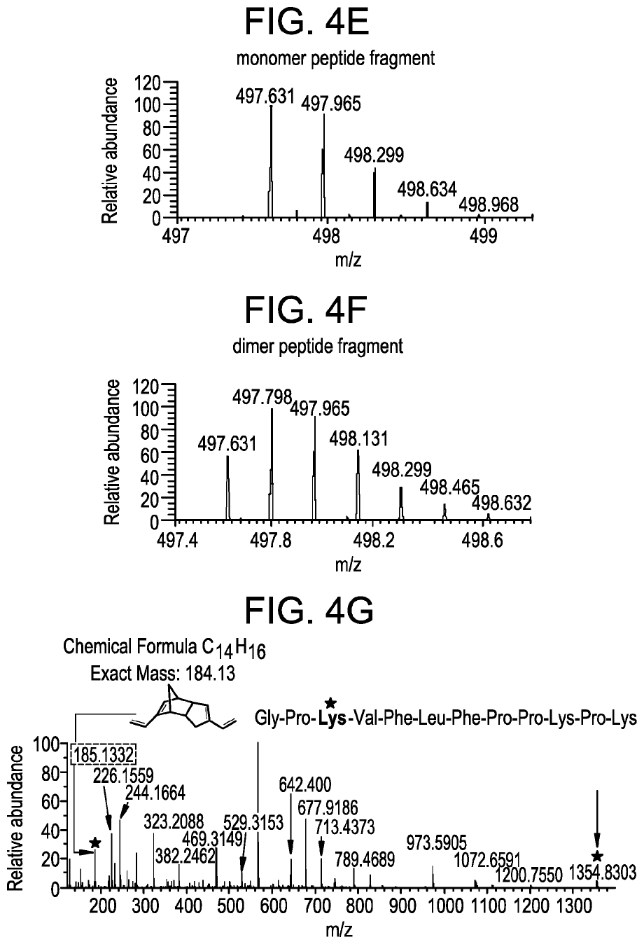

Antibody product 1C1 S239CpK was further evaluated by mass spectrometry to confirm the mechanism of dimerization as formation of a DA adduct. First, antibody was digested with IdeS to remove the Fd region of the antibody by proteolytic cleavage at position 236. Next, the Fc fragment containing the 239 position was further digested with trypsin to generate a 12 amino acid fragment containing S239CpK (FIG. 4a). HPLC analysis showed that two species contained the expected peptide fragments at 97.3% (dimer) and 2.7% (monomer) relative abundance. Dimerized peptide was discerned from monomer peptide by the total mass and charge state. For example, the common peak at ˜497 amu represents a +3 ion, with 0.333 Da isotope spacing for the monomer peptide fragment whereas for the dimer peptide fragment this is a +6 ion, with 0.167 Da isotopic spacing. Evaluation of the MS/MS profile of both monomer and dimer species revealed a species at 185.133 amu unique to the dimer peptide. The mass of this species unique to the dimer peptide corresponds to the DA adduct liberated by fragmentation of CpK at the carbamate bond (FIG. 4g and FIG. 5), thus providing direct evidence of dimer formation.

The ability of CpK to form covalent DA adducts through dimerization appears to be proximity driven for three reasons: 1) no free, unincorporated CpK was detected coupled to antibodies; 2) CpK-containing antibodies did not show high levels of aggregation due to antibody-antibody intermolecular DA reactions; and 3) dimerization occurred at positions S239CpK and P232CpK but not at position K274CpK. The CpK proximity-based self-reaction offers the advantages of being bioorthogonal, spontaneous, and unaffected by canonical amino acids.

The present invention shows that the classic DA reaction can enable new protein engineering strategies. The evaluation of CpK within antibodies allows insight into the biocompatibility, stability, and dimerization properties of this diene. CpK enabled us to evaluate unique properties that have not yet been developed by ncAA platforms: the ability to dimerize in a proximity-dependent manner. In that regard, CpK is analogous to cysteine in its reaction properties (i.e., dimerization) with the added benefit that the DA reaction products are irreversible under physiological conditions. Dimerization of CpK through proximity-driven DA reactions enables a unique bioorthogonal stapling strategy controlled by the diene positions in the folded protein.

### Example 3—Antibody Hinge Region Positions for Heavy Chain Dimer Formation Using CP1 nnAA

Heavy chain amino acids in the hinge region of MAB1VV antibody were mutated to CpK and resulting constructs were evaluated for heavy chain dimerization by SDS-PAGE. The MAB1 antibody comprised the following additional mutations: 1) C226V and C229V mutations in the heavy chain and 2) K409R mutation in the heavy chain. The MAB1VV antibody did not contain native disulfides linking heavy chains together.

Distances between amino acid alpha carbons in the peptide backbone of the antibody were estimated using PyMOL (Schrodinger LLC) and the crystal structure from PDB entry 1FC1. This is shown in Table 2 below.

TABLE 2

| Amino | Number | Distance |
|---|---|---|
| acid | (EU) | (Å)^{a} |
| P | 227 | 10.6 |
| P | 228 | 14.1 |
| C | 229 | 15.5 |
| P | 230 | 9.5 |
| A | 231 | 10.3 |
| P | 232 | 9.9 |
| E | 233 | 9.5 |
| L | 234 | 7.4 |
| L | 235 | 8.7 |
| G | 236 | 10.5 |
| G | 237 | 17.4 |
| P | 238 | 21.1 |
| S | 239 | 18.0 |
| V | 240 | 23.1 |
| F | 241 | 22.2 |

^{a}Estimated from crystal structure data.

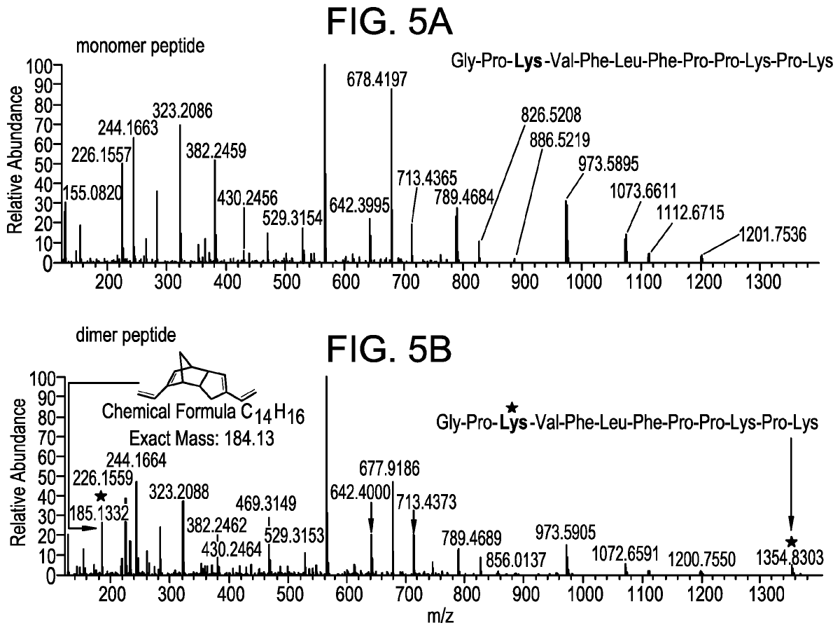

Bands at approximately 100 KDa observed in reduced SDS-PAGE indicated dimerization of antibody heavy chains by CP1 nnAA (FIG. 7). Results demonstrate that CP1 dimerization can occur at multiple positions. These results are summarized in Table 3 below.

TABLE 3

| | Monomer | Day 10 | titer |
|---|---|---|---|
| Mutation | (%) | (mg/L) | Dimerization^{a} |
| C226CP1 | ND | ND | + |
| P227CP1 | 99.7 | 85 | + |
| P228CP1 | 99.5 | 87.5 | + |
| C229CP1 | ND | ND | + |
| P230CP1 | 99.1 | 129.8 | + |
| A231CP1 | 98.9 | 142.6 | + |
| P232CP1 | 99 | 170.5 | ++ |
| E233CP1 | 98.6 | 90.8 | ++ |
| L234CP1 | 98.8 | 110.5 | ++ |
| L235CP1 | 99.2 | 91.3 | ++ |
| G236CP1 | 98.6 | 147.5 | ++ |
| G237CP1 | 98 | 77.7 | ++ |
| P238CP1 | 99.8 | 134.9 | +/− |
| S239CP1 | 99.8 | 73.6 | +++ |
| V240CP1 | 98.9 | 75.6 | +/− |
| F241CP1 | ND | ND | + |

^{a}+ = minor, ++ = moderate, +++ = major, +/− = low and/or inconclusive

### Example 4—Preparation of Heavy Chain Heterodimer Antibodies Using CP1 nnAA and Co-Expression in the Same Cell Culture

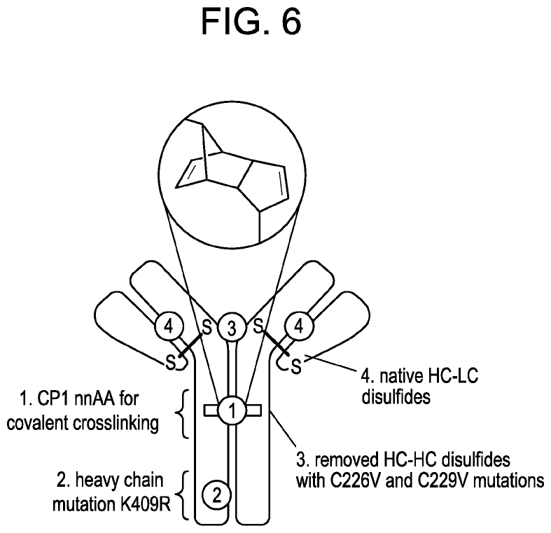

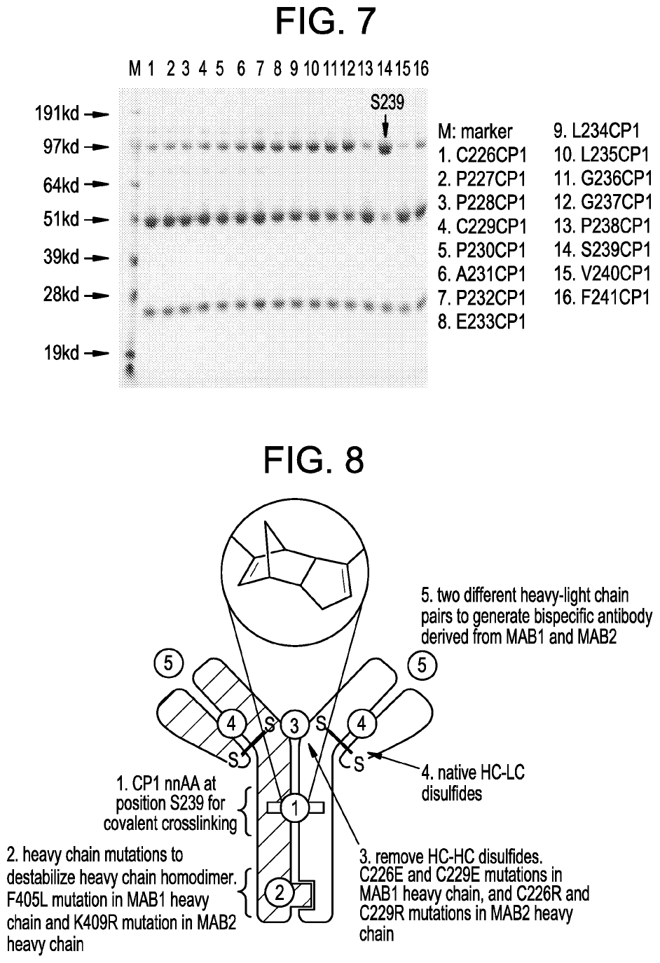

Heavy chain heterodimer antibodies (i.e. monovalent bispecific antibodies) were prepared using the MAB1EE.F405L and MAB2RR.K409R antibodies. MAB1 antibody binds a first human antigen and MAB2 antibody binds a second, different human antigen. For the MAB1 construct, heavy chain disulfides were eliminated by mutation of C226 and C229 to glutamic acid (E) and heavy chain position F405 was mutated to leucine (L). In the MAB2 construct, heavy chain amino acids C226 and C229 were mutated to arginine (R) and heavy chain position K409 was mutated to arginine. These mutations promote heavy chain exchange and non-covalently stabilize the heterodimer. Both MAB1 and MAB2 antibodies contained the S239CP1 mutation to covalently lock heavy chains together after exchange (FIG. 8). Bispecific antibodies were prepared by two methods: 1) transfection and expression of both antibody plasmids in the same cell culture flask (co-transfection) and 2) transfection of each antibody into cells in different culture flask followed by mixing transfected cells and further incubation to complete the expression process (co-culture) (FIG. 9). After treatment of cells with the desired transfection process, antibody expression was conducted as described in Example 1. Antibodies were purified from expression media by either Protein A beads or sequential capture and elution over KappaSelect and LambdaSelect beads. Antibodies were characterized by MS and reduced RP-HPLC as described in Example 1. Concurrent binding studies to first and second antigen proteins were performed on an Octet384 instrument (FortBio). Antibodies at 10 μg/ml in PBS pH 7.2, 3 mg/ml BSA, 0.1% (v/v) Tween20 (assay buffer) were captured on anti-human IgG Fc capture (AHC) biosensors (FortBio). The loaded biosensors were washed with assay buffer to remove any unbound protein. The biosensors were subjected for sequential associations, first with the first antigen at 4 μg/ml followed by incubation with the second antigen at 4 μg/ml. Dissociation was carried out after each association by incubation in assay buffer.

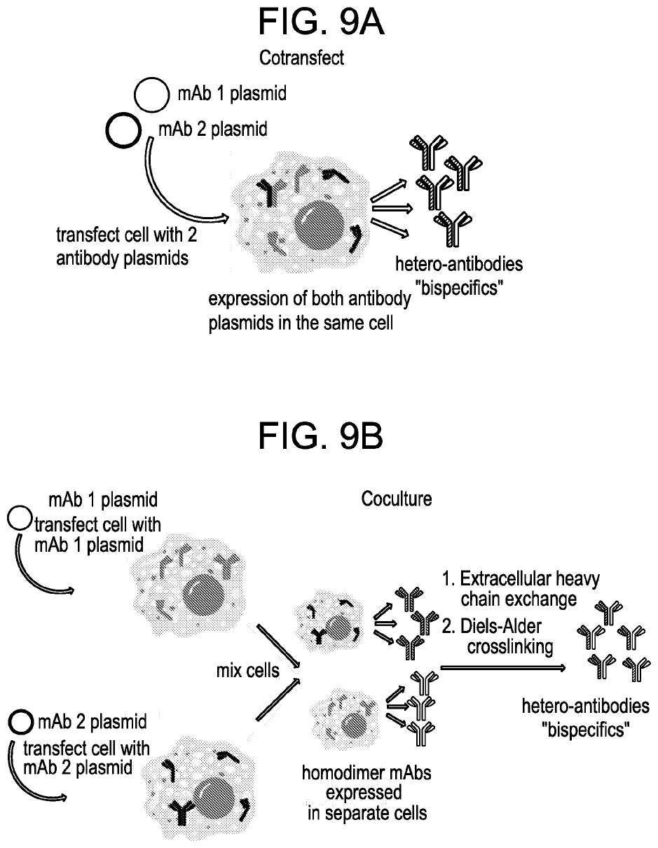

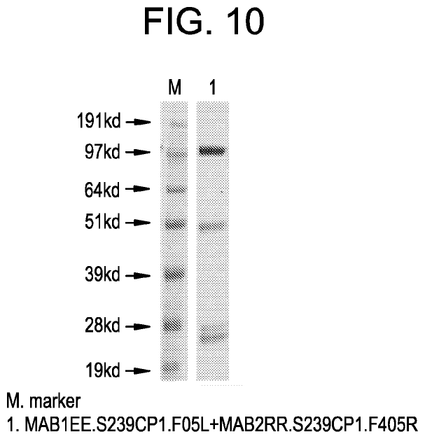

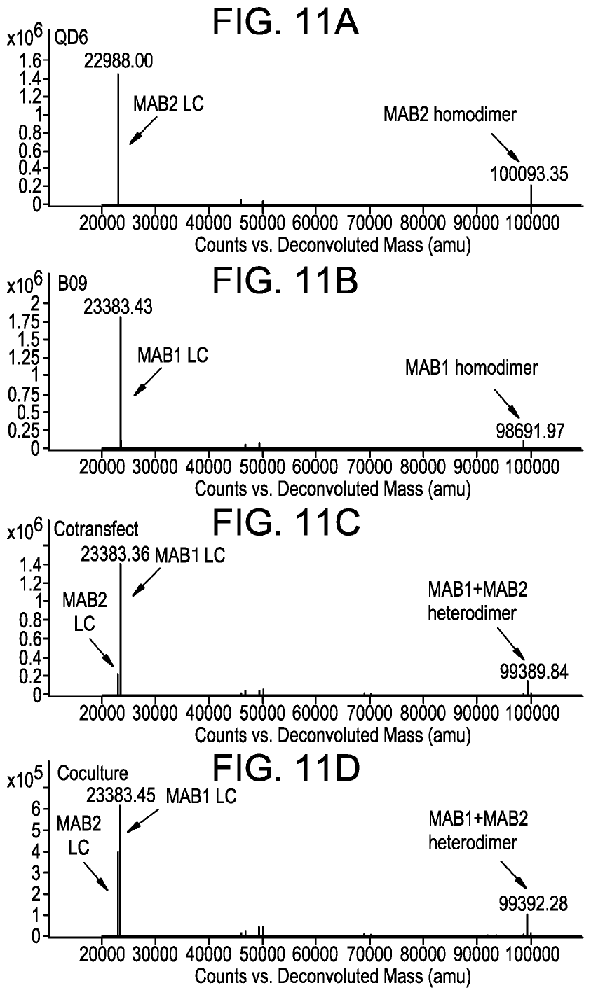

Bispecific antibody yields were similar to yields of monospecific antibody. Reduced SDS-PAGE analysis of bispecific antibody showed that the desired heavy chain dimer formed (FIG. 10) and also indicated the presence of two light chains as expected. Reduced mass spectrometry analysis confirmed formation of heavy chain heterodimers in bispecific antibody constructs, and also the presence of two different light chains with the expected masses for MAB1 and MAB2 light chains (FIG. 11 and FIG. 12). The heterodimer content determined by relative peak heights of heavy chain dimers in the Reduced mass spectrometry analysis is shown below in Table 4.

TABLE 4

| Expression | Heterodimer |
|---|---|
| method | (%)^{a} |
| Cotransfect | 84 |
| Coculture | 88 |

^{a}Determined by relative peak heights of heavy chain dimers in reduced mass spectra.

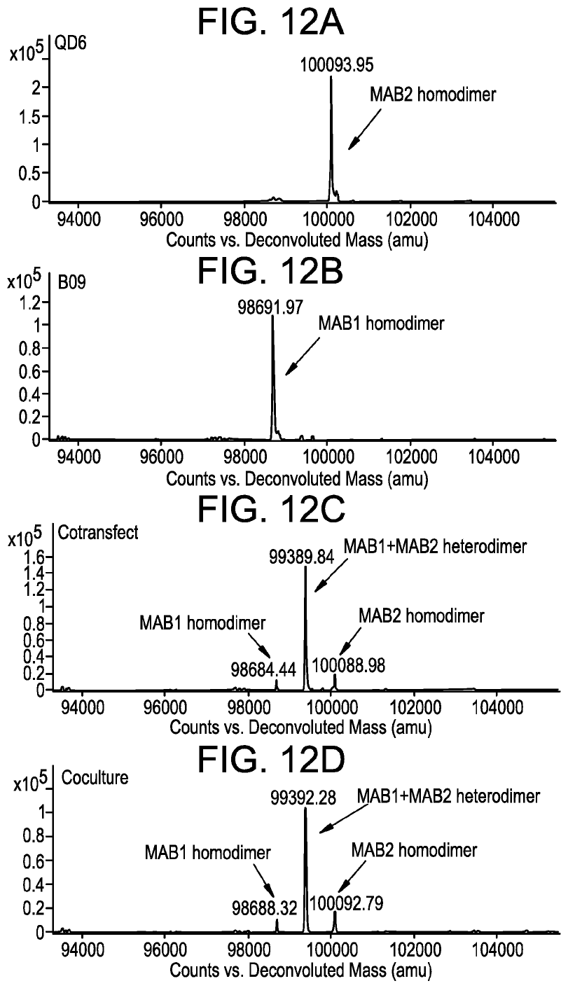

Analysis of bispecific antibody product by reduced RP-HPLC indicated formation of a heavy chain species with a retention time in between MAB1 and MAB2 antibody peaks which is consistent with formation of a heavy chain heterodimer between the two heavy chains (FIG. 13). A summary of the reduced RP-HPLC elution times for homodimer (monospecific) and heterodimer (bispecific) antibodies is provided in Table 5 below.

TABLE 5

| | Elution Time |
|---|---|
| Construct | (min) |
| MAB1EE.S239CP1.F405L | 20.8 |
| MAB2RR.S239CP1.K409R | 21.7 |
| MAB1EE.S239CP1.405L + | 21.2 |
| MAB2RR.S239CP1.K409R | |

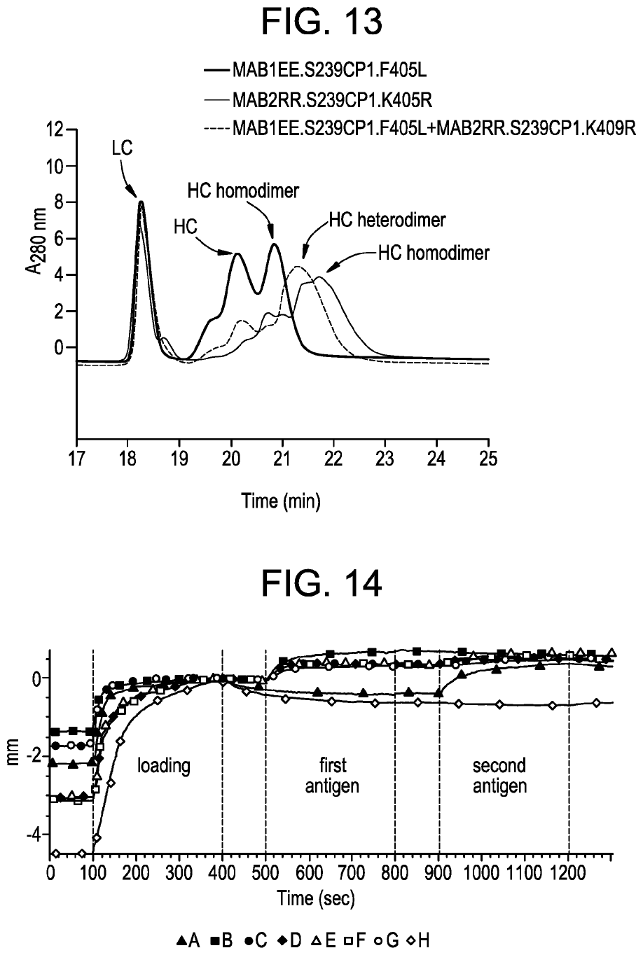

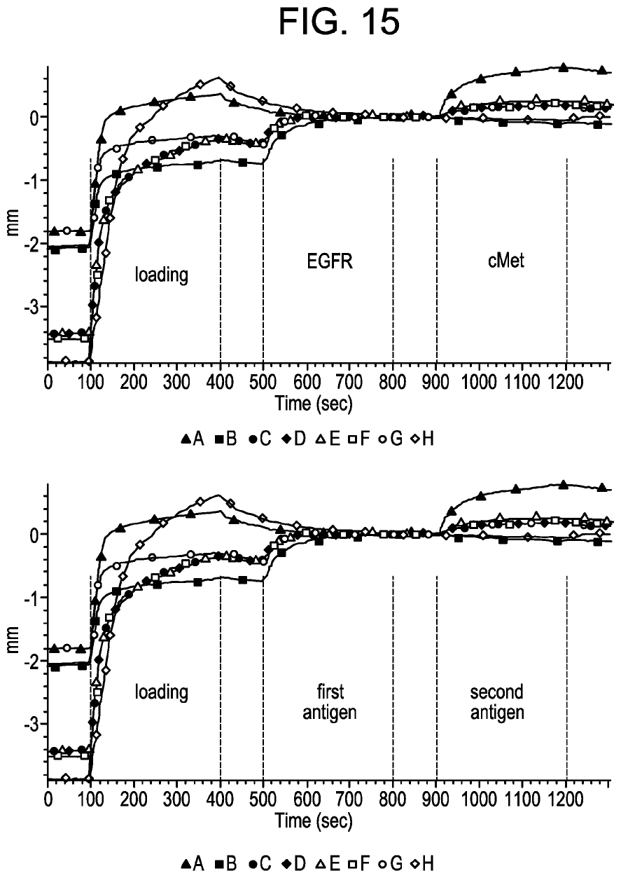

Bispecific antibody function was confirmed for constructs containing CP1 nnAA by antigen binding Octet measurement (FIG. 14 and FIG. 15). Bispecific antibody production method (coculture or cotransfect) did not affect antigen binding with both methods producing bispecific antibodies that had similar binding behavior to the control bispecific antibody prepared without CP1 nnAA.

### REFERENCES

| | Reference | DOI |
|---|---|---|
| Binz | Binz H K, et al., J Mol Biol. 2003 Sep | 10.1016/S0022- |
| 2003 | 12; 332(2): 489-503 | 2836(03)00896-9 |
| Braddock | Braddock M, Expert Opin Investig Drugs. | 10.1517/13543784.16.6.909 |
| 2007 | 2007 Jun; 16(6): 909-17. | |
| Camacho- | Camacho-Soto K, et al., J Am Chem Soc. | 10.1021/ja4130803 |
| Soto 2014 | 2014 136(10): 3995-4002 | |
| Chelur | Chelur D S, Chalfie M. | 10.1073/pnas.0610877104 |
| 2007 | Proc Natl Acad Sci U S A. | |
| | 2007, 104(7): 2283-8. | |
| Cigler | Cigler M, et al., | 10.1002/anie.201706927 |
| 2017 | Angewandte Chemie - International Edition | |
| | 2017, 56, 15737-15741 | |
| Deisenhofer | Deisenhofer J, Biochemistry | 10.1021/bi00512a001 |
| 1981 | 1981, 20, 2361-2370 | |
| Edelman | Edelman G M, et al., | PMID: 5257969 |
| 1969 | Proc Natl Acad Sci U S A. | |
| | 1969; 63(1): 78-85 | |
| Embaby | Embaby A M, et al., Angew Chem Int Ed Engl | 10.1002/anie.201712589 |
| 2018 | 2018, 57, 8022-8026 | |
| Graziano | Graziano and Guptill, Methods Mol Biol., | 10.1385/1-59259-813-7: 071 |
| 2004 | 2004; 283: 71-85 | |
| Holliger | Holliger & Hudson, Nature Biotechnology | 10.1038/nbt1142 |
| 2005 | 2005 23(9): 1126-1136 | |
| Kabat | Kabat E A, Wu T T. J Immunol. | PMID: 1908882 |
| 1991 | 1991 Sep 1; 147(5): 1709-19 | |
| Klein | Klein C, et al., MAbs. | 10.4161/mabs.21379. |
| 2012 | 2012 4(6): 653-63 | |
| Kohl | Kohl A, et al., | 10.1073/pnas.0337680100 |
| 2003 | Proc. Natl. Acad. Sci. USA. | |
| | 2003, 100 (4): 1700-1775 | |
| Kontermann | Kontermann, MAbs | 10.4161/mabs.4.2.19000 |
| 2012 | 2012, 4(2): 182-197 | |
| Kontermann | Kontermann and Brinkmann, | 10.1016/j.drudis.2015.02.008 |
| 2015 | Drug Discovery Today, 2015, 20(7): 838-847 | |
| Mazor | Mazor Y, et al., MAbs. | 10.1080/19420862.2015.1007816 |
| 2015 | 2015; 7(2): 377-89 | |
| Nilsson | Nilsson B, et al., Protein Eng. | PMID: 3507693 |
| 1987 | 1987; 1(2): 107-13 | |
| Oiler-Salvia | Oiler-Salvia B, et al., Angew Chem Int Ed Engl | 10.1002/anie.201712370 |
| 2018 | 2018, 57, 2831-2834 | |
| Parker | Parker M H, et al., Protein Eng Des Sel. | 10.1093/protein/gzi050 |
| 2005 | 2005 Sep; 18(9): 435-44 | |
| Ridgway | Ridgway J B, et al., Protein Eng | PMID: 8844834 |
| 1996 | 1996 9: 617-621 | |
| Silverman | Silverman J, et al., Nat Biotechnol. | 10.1038/nbt1166 |
| 2005 | 2005 Dec; 23(12): 1556-61 | |
| Taylor | Taylor J L, Rohatgi P, Spencer H T, Doyle D F, | 10.1186/1472-6750-10-15 |
| 2010 | Azizi B. BMC Biotechnol. 2010 10: 15. | |
| Vanbrunt | Vanbrunt M P, et al., Bioconjugate Chemistry | 10.1021/acs.bioconjchem.5b00359 |
| 2015 | 2015, 26, 2249-2260. | |
| Wang | Wang N, et al., | 10.1021/jacs.8b01087 |
| 2018 | Journal of the American Chemical Society | |
| | 2018, 140, 4995-4999 | |
| Wikman | Wikman M, et al., Protein Eng Des Sel. | 10.1093/protein/gzh053 |
| 2004 | 2004 May; 17(5): 455-62 | |
| Wu | Wu C Y, et al., Science. 2015 | 10.1126/science.aab4077 |
| 2015 | 350(6258): aab4077 | |
| Xiang | Xiang Z, et al., Nature Methods | 10.1038/nmeth.2595 |
| 2013 | 2013, 10, 885-888 | |
| Xuan | Xuan W, et al., Angew Chem Int Ed Engl | 10.1002/anie.201611841 |
| 2017 | 2017, 56, 5096-5100 | |

1. A conjugate of a first polypeptide and a second polypeptide wherein the link between the first polypeptide and the second polypeptide comprises the moiety:

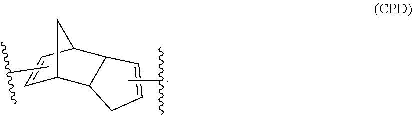

2. A conjugate according to claim 1, wherein at least one link between the group CPD and the polypeptide is of the formula (I*):
^{CPD}*—X¹—O₀₋₁C(O)—*^{PP} (I*)
wherein:
^{CPD}* represents where the link is joined to CPD;
*^{PP} represents where the link is join to the polypeptide;
X¹ represents
i) a saturated or unsaturated branched or unbranched C₁₋₃ alkylene chain, wherein at least one carbon (for example 1, 2 or 3 carbons) is replaced by a heteroatom selected from O, N, S(O)₀₋₃, wherein said chain is optionally, substituted by one or more groups independently selected from oxo, halogen, amino, —C₁₋₃alkylene-N₃, or —C₂₋₅alkynyl; or
ii) together with a carbon from the carbocyclyl or heterocyclyl represents a cyclopropane ring linked to a saturated or unsaturated (in particular saturated) branched or unbranched C₁₋₆ alkylene chain, wherein at least one carbon (for example 1, 2 or 3 carbons) is replaced by a heteroatom selected from O, N, S(O)₀₋₃, wherein said chain is optionally, substituted by one or more groups independently selected from oxo, halogen, amino, —C₁₋₃alkylene-N₃, or —C₂₋₅alkynyl; and
—O₀₋₁C(O)— is linked through a side chain of an amino acid.

3. A conjugate according to claim 1, wherein at least one link between the group CPD and the polypeptide is of the formula (II*): 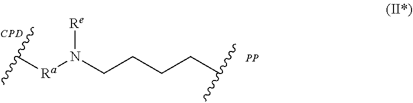
^{CPD}* represents where the link is joined to CPD;
*^{PP} represents where the link is join to the polypeptide;
R^{a} represents:
i) a saturated or unsaturated branched or unbranched C₁₋₈ alkylene chain, wherein at least one carbon (for example 1, 2 or 3 carbons) is replaced by a heteroatom selected from O, N, S(O)₀₋₃, wherein said chain is optionally, substituted by one or more groups independently selected from oxo, halogen, amino; or
ii) together with a carbon from the 5 membered ring represents a cyclopropane ring linked to a saturated or unsaturated branched or unbranched C₁₋₆ alkylene chain, wherein at least one carbon is replaced by a heteroatom selected from O, N, S(O)₀₋₃, wherein said chain is optionally, substituted by one or more groups independently selected from oxo, halogen, amino; and
R* represents H, saturated or unsaturated branched or unbranched C₁₋₈ alkylene chain, wherein one or more carbons are optionally replaced by —O— and the chain is optionally substituted by one or more halogen atoms, N₃ or —C₂₋₅alkynyl.

4. A conjugate according to claim 3, wherein R^{a} is —(CH₂)ₘC(O)—, —CH₂(CH₃)C(O)—, —(CH₂)ₘCH₂OC(O)—, —CHCHCH₂OC(O)—, or —OCH₂CH₂OCO(O)— and m represents 0 or 1.

5. A conjugate according to either claim 3 or claim 4, wherein R^{e} represents H or —CH₂OCH₂CH₂N₃.

6. A conjugate according to any one of claims 3 to 5, wherein CPD and the link are of formula (IIa-CPD):

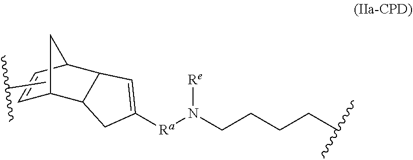

7. A conjugate according to any one of claims 3 to 5, wherein CPD and the link are of formula (IIb-CPD):

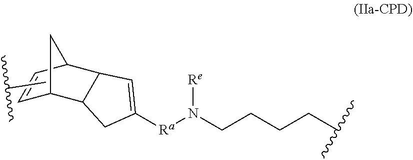

8. A conjugate according to claim 3, wherein CPD and the link to the polypeptide is selected from the group comprising:

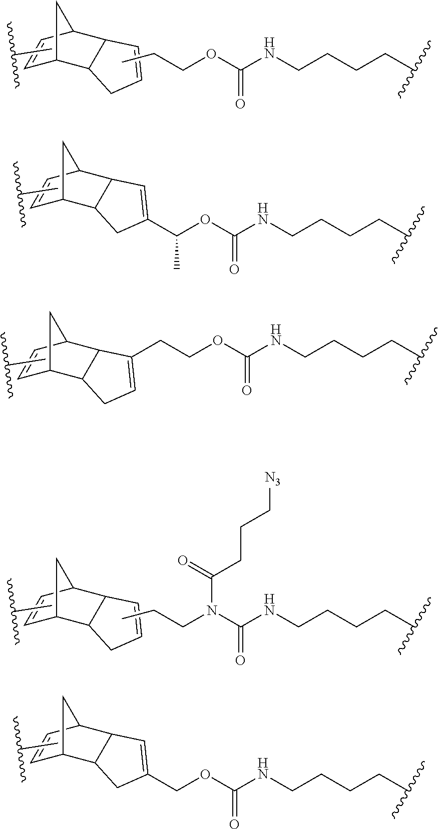

9. A conjugate according to claim 1, wherein at least one link between the group CPD and the polypeptide is of the formula (III*):
^{CPD}*—Bₙ—X³ₘ—Yₚ—Z*—*^{PP} (III*)
wherein:
^{CPD}* represents where the link is joined to CPD;
*^{PP} represents where the link is join to the polypeptide;
n represents 0 or 1;
m represents 0 or 1;
p represents 0 or 1;
B represents C₁₋₆ alkylene, —C₃₋₄ cycloalkylC₁₋₆ alkylene-; wherein a optionally a sugar residue (such as glucose, glucosamine, galactose, galactosamine, lactose, mannose, and fructose) is contained in the alkylene chain of any one of the same, and wherein the alkylene chain of any one of said variables defined for B bears optionally bears one or two substituents independently selected from an N- and O-linked sugar residue (such as glucose, glucosamine, galactose, galactosamine, lactose, mannose, and fructose);
X³ represents —(R¹)NC(O)—, —C(O) N(R¹)—, —OC(O)—, —OC(O)N—;
R¹ represents H or —CH₂OCH₂CH₂R²;
R² represents —N₃, C₂₋₅ alkynyl, or halogen, such as iodo;
Y represents —(OCH₂)_{q}C₂₋₆alkylene, or —C₂₋₆ alkylene optionally substituted with —NR³R⁴;
wherein q is 1 to 7000;
Z* is —C(O)O—, —NC(O)—, triazolyl, —S—, or —NHC(O)—.

10. A conjugate according to claim 1, wherein at least one link between the group CPD and the polypeptide is selected from:

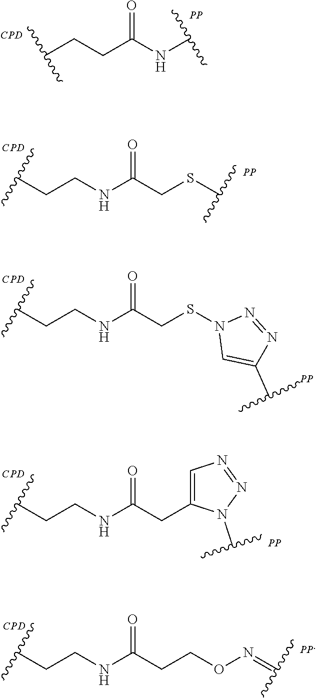

11. A conjugate according to any one of claims 1 to 10, wherein the link between the group CPD and the first polypeptide and the link between the group CPD and the second polypeptide are the same.

12. A conjugate according to any one of claims 1 to 11, wherein the CPD moiety is linked to the first and second polypeptide through a first amino acid side chain on the first polypeptide and a second amino acid side chain on the second polypeptide.

13. A conjugate according to any one of claims 1 to 12, wherein the first and second polypeptides are different.

14. A conjugate according to any one of claims 1 to 12, wherein the first and second polypeptides are identical.

15. A conjugate according to any one of claims 1 to 13, wherein the first or second polypeptide is or comprises a binding member.

16. A conjugate according to claim 15, wherein the binding member is an antibody molecule.

17. A conjugate according to claim 15 or 16, wherein the first polypeptide is or comprises a binding molecule and the second polypeptide is or comprises a synthetic IgG-binding domain.

18. A conjugate according to any one of claims 1 to 14, wherein the conjugate is a split protein.

19. A conjugate according to claim 18, wherein the split protein is selected from the group consisting of a split chimeric antigen receptor, a split kinase, a split transcription factor, and a split caspase.

20. An antibody molecule comprising the conjugate of any one of claims 1 to 14.

21. An antibody molecule according to claim 20, wherein the first polypeptide comprises a first heavy chain region and the second polypeptide comprises a second heavy chain region, and wherein the link between the first heavy chain region and second heavy chain region comprises the CPD moiety.

22. An antibody molecule according to claim 21, wherein the link between the first heavy chain region and second heavy chain region comprising the CPD moiety is located at any one of positions 227, 228, 229, 230, 231, 232, 233, 234, 235, 236, 237, 238, 239, 240 and 241 of the first and second heavy chain regions, wherein the amino acid residue positions are numbered according to EU numbering.

23. An antibody molecule according to claim 22, wherein the link between the first heavy chain region and second heavy chain region comprising the CPD moiety is located at position 239 of the first and second heavy chain regions, wherein the amino acid residue position is numbered according to EU numbering.

24. An antibody molecule according to claim 23, wherein the antibody molecule comprises a first and second heavy chain region, wherein one or both of the first and second heavy chain regions has a residue other than cysteine at positions 226 and 229, wherein the amino acid residue position is numbered according to EU numbering.

25. An antibody molecule according to claim 24, wherein one of the first and second heavy chain regions comprises a positively charged amino acid at positions 226 and 229, and the other heavy chain region comprises a negatively charged amino acid at positions 226 and 229 wherein the amino acid residue position is numbered according to EU numbering.

26. An antibody molecule according to claim 25, wherein the antibody molecule comprises one or more mutations that destabilise the formation of a homodimeric antibody molecule.

27. An antibody molecule according to claim 26, wherein one of the first and second heavy chain regions comprises a glutamic acid (E) residue at positions 226 and 229 and a leucine (L) residue at position 405, and the other heavy chain comprises an arginine (R) residue at positions 226, 229 and 409, wherein the amino acid residue positions are numbered according to EU numbering.

28. An antibody molecule according to claim 20, wherein the first polypeptide comprises a first heavy chain and the second polypeptide comprises a first light chain region, wherein the link between the first heavy chain region and first light chain region comprises the CPD moiety.

29. An antibody molecule according to claim 28, wherein the link between the first heavy chain region and second light chain region comprising the CPD moiety is located between an amino acid in the VH domain of the first heavy chain region and an amino acid in the VL region of the second light chain region.

30. An antibody molecule according to claim 29, wherein the link between the first heavy chain region and second light chain region comprising the CPD moiety is located at position 39 in the heavy chain variable region and position 42 in the light chain variable region, wherein the amino acid residue positions are numbered according to Kabat numbering.

31. A method of conjugating a first polypeptide and a second polypeptide wherein the first polypeptide and the second polypeptide each comprise the moiety: 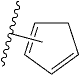
(cyclopentadienyl, CP), where the conjugating involves a Diel-Alder reaction between the cyclopentadienyl moieties.

32. A method according to claim 31, wherein the cyclopentadienyl group is incorporated into the first and/or second polypeptides via the addition of a linker to an amino acid residue in the first and/or second polypeptides, for example where the amino acid is a cysteine or lysine.

33. A method according to claim 31 or claim 32, wherein the reaction is performed at a temperature in the range 0° C. to 70° C.

34. A method according to any one of claims 31 to 33, wherein the reaction is performed in aqueous solvent.

35. A method according to claim 31, wherein at least one of the cyclopentadienyl groups is contained in a non-natural amino acid, for example a non-natural amino acid derived from lysine, cysteine, selenocysteine, aspartic acid, glutamic acid, serine, threonine, glycine, and tyrosine.

36. A method according to claim 35, wherein the cyclopentadienyl group is in a side chain of the amino acid.

37. A method according to claim 35 or claim 36, wherein the method comprises expressing one or more nucleic acids encoding the first and second polypeptide in one or more host cells, adding the non-natural amino acid comprising a CP moiety to the one or more host cells under conditions sufficient to incorporate the CP moiety into the first and second polypeptide, culturing the one or more host cells under conditions that allow a Diel-Alder reaction to occur between the CP moieties to produce a conjugate between the first and second polypeptide, and optionally isolating and/or purifying the conjugate.

38. A method according to claim 37, wherein the method comprises expressing the one or more nucleic acids encoding the first and second polypeptide in the same host cell.

39. A method according to claim 38, wherein the method comprises expressing a first nucleic acid encoding the first polypeptide in a first host cell and expressing a second nucleic acid encoding the second polypeptide in a second host cell, and wherein the Diel-Alder reaction occurs between the CP moieties outside the host cells.

40. A method according to any one of claims 31 to 39, wherein the conjugate is an antibody molecule.

41. A method according to any one of claims 32 to 40, wherein a cyclopentadienyl group is located at a first amino acid residue in the first polypeptide and a cyclopentadienyl group is located at a second amino acid residue in the second polypeptide, such that the distance between the α-carbons of the first and second amino acids in an assembled protein structure is:
a) less than 50 Å;
b) less than 30 Å; or
c) less than 20 Å,
wherein the assembled protein structure is a crystal structure that comprises the first polypeptide in covalent or non-covalent association with the second polypeptide but does not contain the CPD moiety.

42. A method according to any one of claims 32 to 41, wherein a cyclopentadienyl group is located at a first amino acid residue in the first polypeptide and a cyclopentadienyl group is located at a second amino acid residue in the second polypeptide, such that the distance between the α-carbons of the first and second amino acids in an assembled protein structure is:
a) greater than 5 Å;
b) greater than 10 Å; or
c) greater than 15 Å,
wherein the assembled protein structure is a crystal structure that comprises the first polypeptide in covalent or non-covalent association with the second polypeptide but does not contain the CPD moiety.

43. A method according to any one of claims 32 to 42, wherein a cyclopentadienyl group is located at a first amino acid residue in the first polypeptide and a cyclopentadienyl group is located at a second amino acid residue in the second polypeptide, such that the native side chain of the first amino acid residue and the native side chain of the second amino acid residue are orientated towards each other in the assembled protein structure, and wherein the assembled protein structure is a crystal structure that comprises the first polypeptide in covalent or non-covalent association with the second polypeptide but does not contain the CPD moiety.

44. The conjugate formed by the method of any one of claims 31 to 43.
